(19) United States
(12) Patent Application Publication
(10) Pub. No.: US 2023/0241178 A1
(43) Pub. Date: Aug. 3, 2023

(54) LONG ACTING GLP-1/GIP DUAL AGONISTS

(71) Applicant: SUN PHARMACEUTICAL INDUSTRIES LIMITED, Mumbai, Maharashtra (IN)

(72) Inventors: Rajamannar THENNATI, Vadodara, Gujarat (IN); Vinod Sampatrao BURADE, Vadodara, Gujarat (IN); Muthukumaran NATARAJAN, Vadodara, Gujarat (IN); Dhiren Rameshchandra JOSHI, Vadodara, Gujarat (IN); Manish Harendraprasad GANDHI, Vadodara, Gujarat (IN); Chandulal Thakarshibhai JIVANI, Vadodara, Gujarat (IN); Abhishek TIWARI, Kota, Rajasthan (IN); Krunal Harishbhai SONI, Vadodara, Gujarat (IN)

(21) Appl. No.: 18/002,478

(22) PCT Filed: Jun. 21, 2021
(86) PCT No.: PCT/IB2021/055457

§ 371 Date: Dec. 19, 2022

(30) Foreign Application Priority Data
Jun. 22, 2020 (IN) 202021026360
Jan. 20, 2021 (IN) 202121002838

(51) Int. Cl. A61K 38/26; A61K 47/54; A61P 3/10; A61P 3/04

(52) U.S. Cl. CPC A61K 38/26; A61K 47/542; A61P 3/10; A61P 3/04

(57) ABSTRACT

The present invention relates to long acting glucagon-like peptide-1 and human glucose-dependent insulinotropic polypeptide (GIP) agonist polypeptides which may be useful for treating type 2 diabetes mellitus (T2D), diabetes with obesity, obesity and hyperlipidemia.

### FIELD OF THE INVENTION

The present invention relates to a long acting glucagon-like peptide-1 and human glucose-dependent insulinotropic polypeptide/Gastro Intestinal Peptide (GIP) agonist polypeptide which may be useful for treating type 2 diabetes mellitus (T2D), diabetes with obesity, obesity and hyperlipidemia.

### BACKGROUND OF THE INVENTION

Treatment of type 2 diabetes mellitus (T2DM) with glucagon-like peptide-1 receptor agonists (GLP-1RAs) leads to improved glycaemic control, reduced body weight, and improvement in several cardiovascular risk factors. These benefits are mediated by the glucagon-like peptide-1 receptor (GLP-1R), a member of the class B family of G protein-coupled receptors, that is expressed in pancreatic beta-cells, various cell types of the gastrointestinal tract, and neurons throughout both the central (CNS) and the peripheral nervous systems. Activation of GLP-1R signaling by GLP-1RAs improves glucose homeostasis by enhancing glucose-stimulated insulin secretion, delaying gastric emptying, and decreasing plasma glucagon levels, and reduces body weight by activating anorexigenic pathways in the brain. Due to the glucose-dependence of beta-cell activation, GLP-1RAs are not associated with increased risk of hypoglycaemia. While the broad metabolic benefits of GLP-1RAs have established this class in the T2DM treatment paradigm, many patients do not reach their HbA1c/glycemic targets, and weight loss achieved with these agents thus requiring a higher dose, which also increases the GI adverse events, and remains well below what can be attained with bariatric surgery, the most potent clinical intervention for obesity. Thus, there are significant opportunities to improve upon the existing GLP-1RA class of therapeutics.

One emerging approach is to combine foundational GLP-1RA therapy with pharmacological strategies targeting additional pathways implicated in nutrient and energy metabolism, such as glucose-dependent insulinotropic polypeptide (GIP). GIP is an incretin that is secreted from K cells in the upper small intestine, duodenum, in response to food. Postprandial GIP levels are approximately 4-fold higher compared to GLP-1 under normal physiological conditions. GIP is responsible for the majority of the insulinotropic incretin effect in man, and has important additional functions that are distinct from GLP-1. Unlike GLP-1, GIP is both glucagonotropic and insulinotropic in a glycemic-dependent manner, dose-dependently stimulating glucagon secretion under hypoglycemic conditions and insulin under hyperglycemic conditions, glucagon released does facilitate insulin secretion. Although both GIP-receptor (GIPR) and GLP-1R are present in beta-cells, GIPR expression is distributed differently in extra-pancreatic tissues as GIPR is abundant in adipose tissue and is found in many non-overlapping areas of the CNS. GIP is implicated in adipose tissue carbohydrate and lipid metabolism by its actions to regulate glucose uptake, lipolysis, and lipoprotein lipase activity. These findings suggest that pharmacological activation of GIPR may have a therapeutic benefit on peripheral energy metabolism. Recently, uni-molecular, multi-functional peptides that combine GLP-1RA activity with GIP activity have been suggested as new therapeutic agents for glycemic and weight control.

U.S. Pat. No. 9,474,780 discloses dual GLP-1 and GIP receptor agonists including tirzepatide.

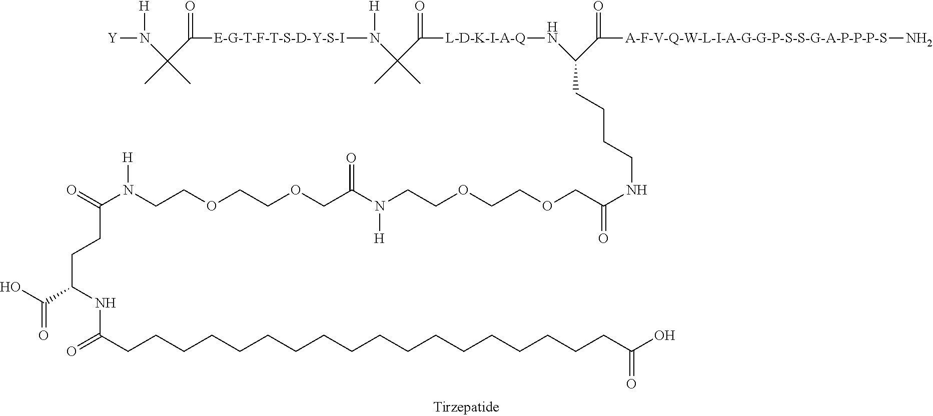

Tirzepatide is under Phase-III clinical studies for T2DM and obesity.

WIPO publication numbers WO201774714A1, WO202023386A1, WO2020023388A1, WO2015067715A2, WO2016111971A1 and WO2013164483A1 disclose GLP-1 R and GIP R dual agonist compounds.

### SUMMARY OF THE INVENTION

The present invention provides a polypeptide or pharmaceutically acceptable salt thereof, comprising an amino acid sequence:

(Seq. ID 1)

Y-X1-E-G-T-F-T-S-D-Y-S-I-X2-L-Xaa15-K-I-A-Xaa19-
X3-Xaa21-F-V-Xaa24-W-L-X4-A-G-G-P-S-S-G-A-P-P-P-
S-X5-X6-X7-X8-X9-X10-X11

wherein X1 is Aib, Ser(OMe) or (D)Ser(OMe);

### X2 is Tyr, Ser(OMe), (D)Ser(OMe) or Aib;

X3 is Gln or Lys; wherein, when X3 is Lys, the side chain amino (ε amino) group of Lys is acylated with a moiety:

{—U—W—Y—Z

wherein U is —C(O)—CH₂—O—(CH₂)₂—O—(CH₂)₂—NH—} wherein} is the point of attachment with group W;
W is selected from a group consisting of —C(O)—NH—(CH₂)ₚ—NH—], —C(O)—C(CH₃)₂—NH—] and —C(O)—CH₂—O—(CH₂)₂—O—(CH₂)₂—NH—], wherein p is 3 or 4 and wherein] is the point of attachment with group Y;
Y is —C(O)—(CH₂)₂—CH(COOH)NH— and — is the point of attachment with the group Z;
Z is —C(O)—(CH₂)ₙ—COOH or —C(O)—(CH₂)ₙ—CH₃ wherein n is an integer from 14 to 20;
and with a proviso that when X3 is Lys and X2 is Aib, then W is not —C(O)—CH₂—O—(CH₂)₂—O—(CH₂)—NH—;

### X4 is Leu, Ile or Glu;

X5 is absent, Arg or Lys; wherein, when X5 is Lys, the side chain amino (ε amino) group of Lys is acylated with a moiety:

{—U′—W′—Y′—Z′

wherein U′ is —C(O)—CH₂—O—(CH₂)₂—O—(CH₂)₂—NH—] wherein} is the point of attachment with group W′;
W′ is selected from a group consisting of —C(O)—NH—(CH₂)_{q}—NH—], —C(O)—C(CH₃)₂—NH—] and —C(O)—CH₂—O—(CH₂)₂—O—(CH₂)₂—NH—], q is 3 or 4 and wherein] is the point of attachment with group Y′;
Y′ is —C(O)—(CH₂)₂—CH(COOH)NH— and — is the point of attachment with the group Z′;
Z′ is —C(O)—(CH₂)ₘ—COOH or —C(O)—(CH₂)ₘ—CH₃ wherein m is an integer from 14 to 20;
X6 is absent or Lys;
X7 is absent or Lys;
X8 is absent or Lys;
X9 is absent or Lys;
X10 is absent or Lys;
X11 is absent or Lys;

### Xaa15 is Asp or Glu;

### Xaa19 is Gln or Ala;

### Xaa21 is Ala or Glu;

### Xaa24 is Gln or Asn.

wherein the acid group of the C terminal amino acid is a free carboxylic acid group or is amidated as C-terminal primary amide;
and with a proviso that at least one of X3 and X5 is Lys.

### ABBREVIATIONS

Aib: 2-Aminoisobutyric acid

### DIPEA: N,N′-Di-isopropylethylamine

### HOBt: 1-Hydroxybenztriazole

### DIPC: N,N′-Di-isopropylcarbodiimide

### THF: Tetrahydrofuran

### DCM: Dichloromethane

### DMAP: 4-Dimethylaminopyridine

### DIC: Diisopropylcarbodiimide

### DMAc: Dimethylacetamide

### DETAILED DESCRIPTION OF THE INVENTION

The present invention provides a stable long acting GLP-1/GIP agonist polypeptide which may be useful for treating type 2 diabetes mellitus (T2D), diabetes with obesity, obesity and hyperlipidemia. The polypeptides of present invention are believed to be long acting, which may not require frequent administration to a patient in need thereof.

Accordingly, in one aspect the present invention provides a polypeptide or pharmaceutically acceptable salt thereof, comprising an amino acid sequence:

(Seq. ID 1)

Y-X1-E-G-T-F-T-S-D-Y-S-I-X2-L-Xaa15-K-I-A-Xaa19-
X3-Xaa21-F-V-Xaa24-W-L-X4-A-G-G-P-S-S-G-A-P-P-P-
S-X5-X6-X7-X8-X9-X10-X11

wherein X1 is Aib, Ser(OMe) or (D)Ser(OMe);

### X2 is Tyr, Ser(OMe), (D)Ser(OMe) or Aib;

X3 is Gln or Lys; wherein, when X3 is Lys, the side chain amino (ε amino) group of Lys is acylated with a moiety:

{—U—W—Y—Z

wherein U is —C(O)—CH₂—O—(CH₂)₂—O—(CH₂)₂—NH—] wherein} is the point of attachment with group W;
W is selected from a group consisting of —C(O)—NH—(CH₂)ₚ—NH—], —C(O)—C(CH₃)₂—NH—] and —C(O)—CH₂—O—(CH₂)₂—O—(CH₂)₂—NH—], wherein p is 3 or 4 and wherein] is the point of attachment with group Y;
Y is —C(O)—(CH₂)₂—CH(COOH)NH— and — is the point of attachment with the group Z;
Z is —C(O)—(CH₂)ₙ—COOH or —C(O)—(CH₂)ₙ—CH₃ wherein n is an integer from 14 to 20;
and with a proviso that when X3 is Lys and X2 is Aib, then W is not —C(O)—CH₂—O—(CH₂)₂—O—(CH₂)₂—NH—];

### X4 is Leu, Ile or Glu;

X5 is absent, Arg or Lys; wherein, when X5 is Lys, the side chain amino (ε amino) group of Lys is acylated with a moiety:

{—U′—W′—Y′—Z′

wherein U′ is —C(O)—CH₂—O—(CH₂)₂—O—(CH₂)₂—NH—] wherein} is the point of attachment with group W′;
W′ is selected from a group consisting of —C(O)—NH—(CH₂)_{q}—NH—], —C(O)—C(CH₃)₂—NH—] and —C(O)—CH₂—O—(CH₂)₂—O—(CH₂)₂—NH—], wherein q is 3 or 4 and wherein] is the point of attachment with group Y′;
Y′ is —C(O)—(CH₂)₂—CH(COOH)NH— and — is the point of attachment with the group Z′;
Z′ is —C(O)—(CH₂)ₘ—COOH or —C(O)—(CH₂)ₘ—CH₃ wherein m is an integer from 14 to 20;
X6 is absent or Lys;
X7 is absent or Lys;
X8 is absent or Lys;
X9 is absent or Lys;
X10 is absent or Lys;
X11 is absent or Lys;

### Xaa15 is Asp or Glu;

### Xaa19 is Gln or Ala;

### Xaa21 is Ala or Glu;

### Xaa24 is Gln or Asn;

wherein the acid group of the C terminal amino acid is a free carboxylic acid group or is amidated as C-terminal primary amide;
and with a proviso that at least one of X3 and X5 is Lys.

In one embodiment of the present invention, X1 is Aib.

In another embodiment of the present invention, X2 is Aib.

In another embodiment of the present invention, X1 and X2 both are Aib.

In another embodiment of the present invention, X1 is Aib and X2 is Ser(OMe) or (D)Ser(OMe).

In another embodiment of the present invention, X1 is Ser(OMe) or (D)Ser(OMe) and X2 is Aib.

In another embodiment of the present invention, X4 is Leu or Ile.

In another embodiment of the present invention, X4 is Ile.

In another embodiment of the present invention, X5 is Lys or Arg.

In another embodiment of the present invention, X3 is Lys and X5 is absent or Arg.

In another embodiment of the present invention, X3 is Gln and X5 is Lys.

In another embodiment of present invention, W is —C(O)—C(CH₃)₂—NH—].

In another embodiment of the present invention, W is —C(O)—NH—(CH₂)ₚ—NH—], wherein p is 3 or 4.

In another embodiment of the present invention, W is —C(O)—NH—(CH₂)₄—NH—].

In another embodiment of the present invention, W is —C(O)—CH₂—O—(CH₂)₂—O—(CH₂)₂—NH—].

In another embodiment of present invention, W′ is —C(O)—C(CH₃)₂—NH—].

In another embodiment of the present invention, W′ is —C(O)—NH—(CH₂)_{q}—NH—], wherein q is 3 or 4.

In another embodiment of the present invention, W′ is —C(O)—NH—(CH₂)₄—NH—].

In another embodiment of the present invention, W′ is —C(O)—CH₂—O—(CH₂)₂—O—(CH₂)₂—NH—].

In another embodiment of the present invention, the C terminal amino acid is amidated as a C-terminal primary amide.

In another embodiment of the present invention, the acid group of the C terminal amino acid is a free carboxylic acid.

In another embodiment of the present invention, n is 16, 17, 18, 19 or 20. In a preferred embodiment n is 18 or 20. In yet another preferred embodiment n is 20. In another preferred embodiment, n is 16 or 18. In yet preferred embodiment, n is 18.

In another embodiment of the present invention, Z is —C(O)—(CH₂)ₙ—COOH and n is 16 or 18.

In another embodiment of the present invention, m is 16, 17, 18, 19 or 20. In a preferred embodiment m is 18 or 20. In yet another preferred embodiment m is 20. In another preferred embodiment, m is 16 or 18. In yet preferred embodiment, m is 18.

In another embodiment of the present invention, Z′ is —C(O)—(CH₂)ₘ—COOH and m is 16 or 18.

In another embodiment of the present invention, W is —C(O)—NH—(CH₂)₄—NH—], Z is —C(O)—(CH₂)ₙ—COOH and n is 18.

In another embodiment of the present invention, W is —C(O)—C(CH₃)₂—NH—], Z is —C(O)—(CH₂)ₙ—COOH and n is 16.

In another embodiment of the present invention, W is —C(O)—C(CH₃)₂—NH—], Z is —C(O)—(CH₂)ₙ—COOH and n is 18.

In another embodiment of the present invention, W is —C(O)—CH₂—O—(CH₂)₂—O—(CH₂)₂—NH—], Z is —C(O)—(CH₂)ₙ—COOH and n is 16.

In another embodiment of the present invention, W is —C(O)—CH₂—O—(CH₂)₂—O—(CH₂)₂—NH—], Z is —C(O)—(CH₂)ₙ—COOH and n is 18.

In another embodiment of the present invention, W′ is —C(O)—NH—(CH₂)₄—NH—], Z′ is —C(O)—(CH₂)ₘ—COOH and m is 18.

In another embodiment of the present invention, W′ is —C(O)—C(CH₃)₂—NH—], Z′ is —C(O)—(CH₂)ₘ—COOH and m is 16.

In another embodiment of the present invention, W′ is —C(O)—C(CH₃)₂—NH—], Z′ is —C(O)—(CH₂)ₘ—COOH and m is 18.

In another embodiment of the present invention, W′ is —C(O)—CH₂—O—(CH₂)₂—O—(CH₂)₂—NH—], Z′ is —C(O)—(CH₂)ₘ—COOH and m is 16.

In another embodiment of the present invention, W′ is —C(O)—CH₂—O—(CH₂)₂—O—(CH₂)₂—NH—], Z′ is —C(O)—(CH₂)ₘ—COOH and m is 18.

In another embodiment of the present invention, X5, X6, X7, X8, X9, X10 and X11 are all absent.

In another embodiment of the present invention, Xaa15 is Asp.

In another embodiment of the present invention, Xaa19 is Gln.

In another embodiment of the present invention, Xaa21 is Ala.

In another embodiment of the present invention, Xaa24 is Gln.

In another embodiment of the present invention, X1 is Aib and X2 is Ser(OMe) or Tyr.

In another embodiment of the present invention, X1 is Aib and X2 is Ser(OMe).

In another embodiment of the present invention, X1 is Aib and X2 is Tyr.

In another embodiment of the present invention, X3 is Gln,

In another embodiment of the present invention, X4 is Leu.

In another embodiment of the present invention, X5 is Lys, wherein the side chain amino (ε amino) group of Lys is acylated with a moiety:

{—U′—W′—Y′—Z′.

In another embodiment of the present invention, W′ is —C(O)—C(CH₃)₂—NH—], Z′ is —C(O)(CH₂)ₘ—COOH and m is 18.

In another embodiment of the present invention, Xaa15 is Glu.

In another embodiment of the present invention, Xaa19 is Ala.

In another embodiment of the present invention, Xaa21 is Glu.

In another embodiment of the present invention, Xaa24 is Asn.

In another embodiment of the present invention, X6, X7, X8, X9, X10 and X11 are all absent.

In another aspect, the present invention provides a polypeptide or pharmaceutically acceptable salt thereof, comprising an amino acid sequence:

(Seq. ID 2)
Y-Aib-E-G-T-F-T-S-D-Y-S-I-Ser(OMe)-L-D-K-I-A-Q-X3-
A-F-V-Q-W-L-X4-A-G-G-P-S-S-G-A-P-P-P-S-X5-X6-X7-
X8-X9-X10-X11,

wherein
X3 is Lys, wherein the side chain amino (ε amino) group of Lys is acylated with a moiety:

{—U—W—Y—Z

wherein U is —C(O)—CH₂—O—(CH₂)₂—O—(CH₂)₂—NH—} wherein} is the point of attachment with group W;
W is selected from a group consisting of —C(O)—NH—(CH₂)ₚ—NH—], —C(O)—C(CH₃)₂—NH—] and —C(O)—CH₂—O—(CH₂)₂—O—(CH₂)₂—NH—], wherein p is 3 or 4 and wherein] is the point of attachment with group Y;
Y is —C(O)—(CH₂)₂—CH(COOH)NH— and — is the point of attachment with the group Z;
Z is —C(O)—(CH₂)ₙ—COOH or —C(O)—(CH₂)ₙ—CH₃ wherein n is an integer from 14 to 20;

### X4 is Ile or Glu;

X5 is absent or Arg;
X6 is absent or Lys;
X7 is absent or Lys;
X8 is absent or Lys;
X9 is absent or Lys;
X10 is absent or Lys;
X11 is absent or Lys;
and wherein the acid group of the C terminal amino acid is a free carboxylic acid group or is amidated as a C-terminal primary amide.

In one embodiment of the present invention, X4 is Ile.

In another embodiment of present invention, W is —C(O)—C(CH₃)₂—NH—].

In another embodiment of the present invention, W is —C(O)—NH—(CH₂)ₚ—NH—], wherein p is 3 or 4.

In another embodiment of the present invention, W is —C(O)—NH—(CH₂)₄—NH—].

In another embodiment of the present invention, W is —C(O)—CH₂—O—(CH₂)₂—O—(CH₂)₂—NH—].

In another embodiment of the present invention, the C terminal amino acid is amidated as a C-terminal primary amide.

In another embodiment of the present invention, n is 16, 17, 18, 19 or 20. In a preferred embodiment n is 18 or 20. In yet another preferred embodiment n is 20. In another preferred embodiment, n is 16 or 18. In yet preferred embodiment, n is 18.

In another embodiment of the present invention, Z is —C(O)—(CH₂)ₙ—COOH and n is 16 or 18.

In another embodiment of the present invention, W is —C(O)—C(CH₃)₂—NH—], Z is —C(O)—(CH₂)ₙ—COOH and n is 16.

In another embodiment of the present invention, W is —C(O)—C(CH₃)₂—NH—], Z is —C(O)—(CH₂)ₙ—COOH and n is 18.

In another embodiment of the present invention, W is —C(O)—CH₂—O—(CH₂)₂—O—(CH₂)₂—NH—], Z is —C(O)—(CH₂)ₙ—COOH and n is 16.

In another embodiment of the present invention, W is —C(O)—CH₂—O—(CH₂)₂—O—(CH₂)₂—NH—], Z is —C(O)—(CH₂)ₙ—COOH and n is 18.

In another embodiment of the present invention, X5, X6, X7, X8, X9, X10 and X11 are all absent.

In another aspect, the present invention provides a polypeptide or pharmaceutically acceptable salt thereof, comprising an amino acid sequence:

(Seq. ID 3)
Y-X1-E-G-T-F-T-S-D-Y-S-I-X2-L-D-K-I-A-Q-X3-A-F-V-
Q-W-L-X4-A-G-G-P-S-S-G-A-P-P-P-S

wherein X1 is Aib; X2 is Ser(OMe) or Aib; X4 is Ile or Glu;
X3 is Lys wherein the side chain amino (ε amino) group of Lys is acylated with a moiety:

### {—U—W—Y—Z

wherein U is —C(O)—CH₂—O—(CH₂)₂—O—(CH₂)₂—NH—} wherein} is the point of attachment with group W;
W is selected from a group consisting of —C(O)—NH—(CH₂)ₚ—NH—], —C(O)—C(CH₃)₂—NH—] and —C(O)—CH₂—O—(CH₂)₂—O—(CH₂)₂—NH—], wherein p is 3 or 4 and wherein] is point of attachment with group Y;
Y is —C(O)—(CH₂)₂—CH(COOH)NH— and — is the point of attachment with the group Z;
Z is —C(O)—(CH₂)ₙ—COOH or —C(O)—(CH₂)ₙ—CH₃ wherein n is an integer from 14 to 20;
and wherein the acid group of the C terminal amino acid is a free carboxylic acid group or is amidated as a C-terminal primary amide;
with a proviso that when X2 is Aib, then W is not —C(O)—CH₂—O—(CH₂)₂—O—(CH₂)₂—NH—].

In one embodiment of the present invention, X2 is Aib and X4 is Ile.

In another embodiment of present invention, W is —C(O)—C(CH₃)₂—NH—].

In another embodiment of the present invention, W is —C(O)—NH—(CH₂)ₚ—NH—] and wherein p is 3 or 4.

In another embodiment of the present invention, W is —C(O)—NH—(CH₂)₄—NH—].

In another embodiment of the present invention, W is —C(O)—CH₂—O—(CH₂)₂—O—(CH₂)₂—NH—].

In another embodiment of the present invention, the C terminal amino acid is amidated as C-terminal primary amide.

In another embodiment of the present invention, n is 16, 17, 18, 19 or 20. In a preferred embodiment n is 18 or 20. In yet another preferred embodiment n is 20. In another preferred embodiment, n is 16 or 18. In yet preferred embodiment, n is 18.

In another embodiment of the present invention, Z is —C(O)—(CH₂)ₙ—COOH and n is 16 or 18.

In another embodiment of the present invention, W is —C(O)—NH—(CH₂)₄—NH—], Z is —C(O)—(CH₂)ₙ—COOH and n is 18.

In another embodiment of the, W is —C(O)—C(CH₃)₂—NH—], Z is —C(O)—(CH₂)ₙ—COOH and n is 16.

In another embodiment of the present invention, W is —C(O)—C(CH₃)₂—NH—], Z is —C(O)—(CH₂)ₙ—COOH and n is 18.

In another embodiment of the present invention, X2 is Ser(OMe) and X4 is Ile.

In another embodiment of the present invention, W is —C(O)—C(CH₃)₂—NH—], Z is —C(O)—(CH₂)ₙ—COOH and n is 16.

In another embodiment of the present invention, W is —C(O)—CH₂—O—(CH₂)₂—O—(CH₂)₂—NH—], Z is —C(O)—(CH₂)ₙ—COOH and n is 16.

In another embodiment of the present invention, W is —C(O)—CH₂—O—(CH₂)₂—O—(CH₂)₂—NH—], Z is —C(O)—(CH₂)ₙ—COOH and n is 18.

In another aspect, the present invention provides a polypeptide or pharmaceutically acceptable salt thereof, comprising an amino acid sequence:

(Seq. ID 4)
Y-Aib-E-G-T-F-T-S-D-Y-S-I-Aib-L-D-K-I-A-Q-X3-A-F-
V-Q-W-L-Ile-A-G-G-P-S-S-G-A-P-P-P-S

wherein X3 is Lys wherein the side chain amino (ε amino) group of Lys is acylated with a moiety:

{—U—W—Y—Z

wherein U is —C(O)—CH₂—O—(CH₂)₂—O—(CH₂)₂—NH—} wherein} is the point of attachment with group W;
W is selected from a group consisting of —C(O)—NH—(CH₂)ₚ—NH—] or —C(O)—C(CH₃)₂—NH—],
wherein p is 3 or 4 and wherein] is the point of attachment with group Y;
Y is —C(O)—(CH₂)₂—CH(COOH)NH— and — is the point of attachment with the group Z;
Z is —C(O)—(CH₂)ₙ—COOH or —C(O)—(CH₂)ₙ—CH₃ wherein n is an integer from 14 to 20;
and wherein the acid group of the C terminal amino acid is a free carboxylic acid group or is amidated as a C-terminal primary amide.

In another embodiment of present invention, W is —C(O)—C(CH₃)₂—NH—].

In another embodiment of the present invention, W is —C(O)—NH—(CH₂)ₚ—NH—] and wherein p is 3 or 4.

In another embodiment of the present invention, W is —C(O)—NH—(CH₂)₄—NH—].

In another embodiment of the present invention, the C terminal amino acid is amidated as a C-terminal primary amide.

In another embodiment of the present invention, n is 16, 17, 18, 19 or 20. In a preferred embodiment n is 18 or 20. In yet another preferred embodiment n is 20. In another preferred embodiment, n is 16 or 18. In yet preferred embodiment, n is 18.

In another embodiment of the present invention, Z is —C(O)—(CH₂)ₙ—COOH and n is 16 or 18.

In another embodiment of the present invention, W is —C(O)—NH—(CH₂)₄—NH—], Z is —C(O)—(CH₂)ₙ—COOH and n is 18.

In another embodiment of the present invention, W is —C(O)—C(CH₃)₂—NH—], Z is —C(O)—(CH₂)ₙ—COOH and n is 16.

In another embodiment of the present invention, W is —C(O)—C(CH₃)₂—NH—], Z is —C(O)—(CH₂)ₙ—COOH and n is 18.

In another aspect, the present invention provides a polypeptide or pharmaceutically acceptable salt thereof, comprising an amino acid sequences selected from:

i)
Tyr Aib Glu Gly Thr Phe Thr Ser Asp Tyr Ser Ile
Aib Leu Asp Lys Ile Ala Gln X3 Ala Phe Val Gln
Trp Leu Ile Ala Gly Gly Pro Ser Ser Gly Ala Pro
Pro Pro Ser;
ii)
Tyr Aib Glu Gly Thr Phe Thr Ser Asp Tyr Ser Ile
D-Ser-(OMe) Leu Asp Lys Ile Ala Gln X3 Ala Phe
Val Gln Trp Leu Ile Ala Gly Gly Pro Ser Ser Gly
Ala Pro Pro Pro Ser;
iii)
Tyr Aib Glu Gly Thr Phe Thr Ser Asp Tyr Ser Ile
Ser(OMe) Leu Asp Lys Ile Ala Gln X3 Ala Phe Val
Gln Trp Leu Ile Ala Gly Gly Pro Ser Ser Gly Ala
Pro Pro Pro Ser;
iv)
Tyr Aib Glu Gly Thr Phe Thr Ser Asp Tyr Ser Ile
Aib Leu Asp Lys Ile Ala Gln X3 Ala Phe Val Gln
Trp Leu Ile Ala Gly Gly Pro Ser Ser Gly Ala Pro
Pro Pro Ser Arg;
v)
Tyr Aib Glu Gly Thr Phe Thr Ser Asp Tyr Ser Ile
Tyr Leu Glu Lys Ile Ala Ala Tyr Glu Phe Val Asn
Trp Leu Leu Ala Gly Gly Pro Ser Ser Gly Ala Pro
Pro Pro Ser X5;
vi)
Tyr Aib Glu Gly Thr Phe Thr Ser Asp Tyr Ser Ile
Ser(OMe) Leu Glu Lys Ile Ala Ala Gln Glu Phe
Val Asn Trp Leu Leu Ala Gly Gly Pro Ser Ser Gly
Ala Pro Pro Pro Ser X5;
vii)
Tyr D-Ser(OMe) Glu Gly Thr Phe Thr Ser Asp Tyr
Ser Ile Aib Leu Asp Lys Ile Ala Gln X3 Ala Phe
Val Gln Trp Leu Ile Ala Gly Gly Pro Ser Ser
Gly Ala Pro Pro Pro Ser;
and
viii)
Tyr Ser(OMe) Glu Gly Thr Phe Thr Ser Asp Tyr
Ser Ile Aib Leu Asp Lys Ile Ala Gln X3 Ala Phe
Val Gln Trp Leu Ile Ala Gly Gly Pro Ser Ser
Gly Ala Pro Pro Pro Ser.

wherein, X3 and X5 have the same meaning as set forth above;
and wherein the acid group of the C terminal amino acid is a free carboxylic acid group or is amidated as a C-terminal primary amide.

In another aspect, the present invention provides a polypeptide or pharmaceutically acceptable salt thereof comprising an amino acid sequence selected from the group consisting of:

i)

(SEQ ID NO 5)

Tyr Aib Glu Gly Thr Phe Thr Ser Asp Tyr Ser Ile
Aib Leu Asp Lys Ile Ala Gln Lys Ala Phe Val Gln
Trp Leu Ile Ala Gly Gly Pro Ser Ser Gly Ala Pro
Pro Pro Ser-NH₂;
ii)

(SEQ ID NO 9)

Tyr Aib Glu Gly Thr Phe Thr Ser Asp Tyr Ser Ile
D-Ser-(OMe) Leu Asp Lys Ile Ala Gln Lys Ala Phe
Val Gln Trp Leu Ile Ala Gly Gly Pro Ser Ser Gly
Ala Pro Pro Pro Ser-NH₂;
iii)

(SEQ ID NO 10)

Tyr Aib Glu Gly Thr Phe Thr Ser Asp Tyr Ser Ile
Ser(OMe) Leu Asp Lys Ile Ala Gln Lys Ala Phe
Val Gln Trp Leu Ile Ala Gly Gly Pro Ser Ser Gly
Ala Pro Pro Pro Ser-NH₂;
iv)

(SEQ ID NO 11)

Tyr Aib Glu Gly Thr Phe Thr Ser Asp Tyr Ser Ile
Aib Leu Asp Lys Ile Ala Gln Lys Ala Phe Val Gln
Trp Leu Ile Ala Gly Gly Pro Ser Ser Gly Ala Pro
Pro Pro Ser Arg;
v)

(SEQ ID NO 12)

Tyr Aib Glu Gly Thr Phe Thr Ser Asp Tyr Ser Ile
Tyr Leu Glu Lys Ile Ala Ala Gln Glu Phe Val Asn
Trp Leu Leu Ala Gly Gly Pro Ser Ser Gly Ala Pro
Pro Pro Ser Lys-NH₂;
vi)

(SEQ ID NO 13)

Tyr Aib Glu Gly Thr Phe Thr Ser Asp Tyr Ser Ile
Ser(OMe) Leu Glu Lys Ile Ala Ala Gln Glu Phe
Val Asn Trp Leu Leu Ala Gly Gly Pro Ser Ser Gly
Ala Pro Pro Pro Ser Lys-NH₂;
vii)

(SEQ ID NO 6)

Tyr D-Ser(OMe) Glu Gly Thr Phe Thr Ser Asp Tyr
Ser Ile Aib Leu Asp Lys Ile Ala Gln Lys Ala Phe
Val Gln Trp Leu Ile Ala Gly Gly Pro Ser Ser Gly
Ala Pro Pro Pro Ser-NH₂;
and
viii)

(SEQ ID NO 7)

Tyr Ser(OMe) Glu Gly Thr Phe Thr Ser Asp Tyr
Ser Ile Aib Leu Asp Lys Ile Ala Gln Lys Ala Phe
Val Gln Trp Leu Ile Ala Gly Gly Pro Ser Ser Gly
Ala Pro Pro Pro Ser-NH₂.

In another aspect, the present invention provides a polypeptide or pharmaceutically acceptable salt thereof, selected from the representative compounds as disclosed in the Table 1.

In the embodiments of the present invention, the groups U, W, Y and Z in the moiety

{—U—W—Y—Z

or the groups U′, W′, Y′ and Z′ in the moiety

{—U′—W′—Y′—Z′

have meaning as defined in this specification and should not be interpreted as or mixed with the single letter code of the amino acids;
and wherein, the group —U—W—Y—Z and/or —U′—W′—Y′—Z′ is selected from the representative structures of Moiety A, B, C, D and E as disclosed in Table 2.

Ser(OMe) as described herein in the specification is amino acid serine, preferably the L isomer, with its hydroxyl group methylated and has following structure:

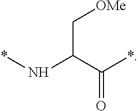

Wherever applicable, (D)Ser(OMe) refers to the D isomer of Ser(OMe).

Tyr-(OEt) as described herein in the specification is amino acid tyrosine, preferably the L isomer, with the hydroxyl group ethylated and has the following structure (* denotes points of attachment to adjacent residues).

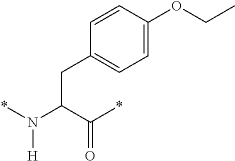 [SMILES: *NC(Cc1ccc(OCC)cc1)C(*)=O]

Wherever applicable, (D)Tyr(OEt) refers to the D isomer of Tyr(OEt).

The polypeptide sequences mentioned in the specification are represented either by the single letter code or three letter code of the amino acids as approved by IUPAC.

Unless stated otherwise, the specification intends to cover both L and D isomers of the amino acids in the sequence. However, in preferred embodiments, all the amino acids are in “L” configuration unless indicated otherwise.

A “Pharmaceutically acceptable salt” according to the invention includes an acid addition salt formed with either organic or inorganic acids. Suitable pharmaceutically acceptable salts of the compounds of the invention include acid addition salts which may be salts of inorganic acids such as hydrochloric acid, hydrobromic acid, phosphoric acid, and the like or of organic acids such as, for example, acetic acid, benzenesulfonic acid, methanesulfonic acid, benzoic acid, citric acid, lactic acid, fumaric acid, succinic acid, adipic acid, pimelic acid, suberic acid, azelaic acid, malic acid, tartaric acid, amino acids such as glutamic acid or aspartic acid, and the like. The pharmaceutically acceptable acid addition salts of the present invention include salts formed with the addition of one or more equivalents of acids, for example, monohydrochloride, dihydrochloride salts, etc. Salts can be prepared by any process under the purview of an ordinary person skilled in the art (see Berge et al., J. Pharm. Sci. 1977, 66, 1-19; and Handbook of Pharmaceutical Salts, Properties, and Use; Stahl and Wermuth, Ed.; Wiley-VCH and VHCA: Zurich, Switzerland, 2002).

Table 1 provides some of the representative compounds of the present invention.

TABLE 1
Representative polypeptide compounds of present disclosure

| Compd | | SEQ |
|---|---|---|
| No. | Structure | ID |
| 1 | 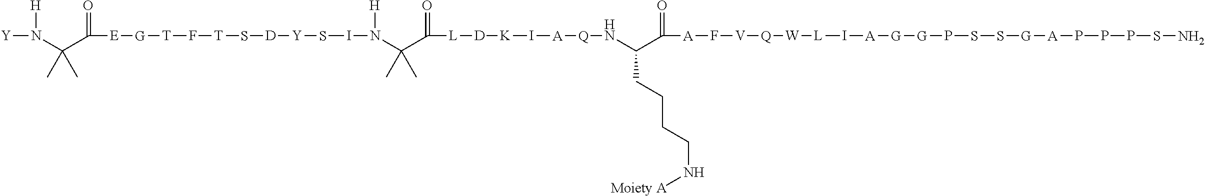 | Seq ID: 05 |
| 2 | 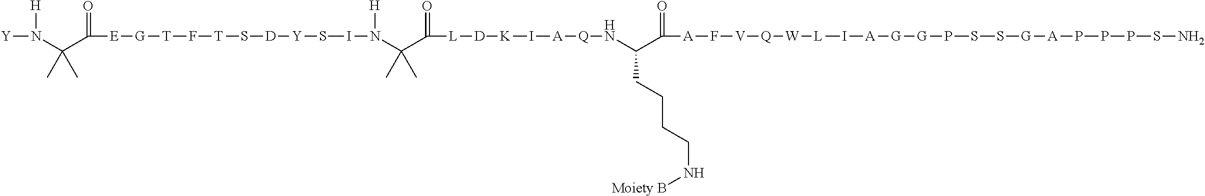 | Seq ID: 05 |
| 3 | 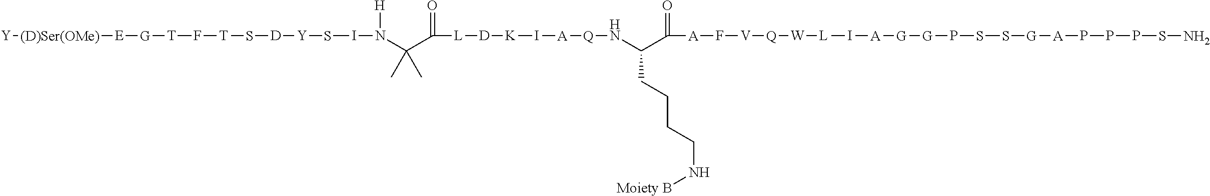 | Seq ID: 06 |
| 4 | 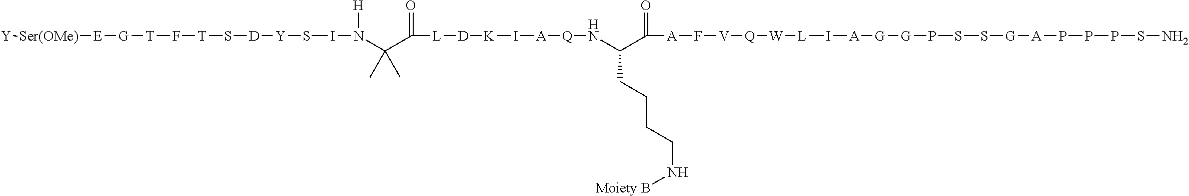 | Seq ID: 07 |
| 5 | 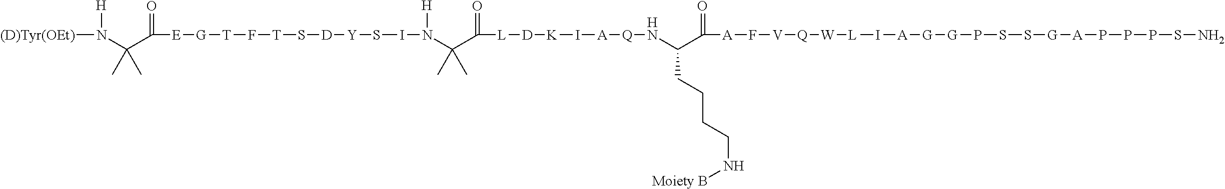 | Seq ID: 08 |
| 6 | 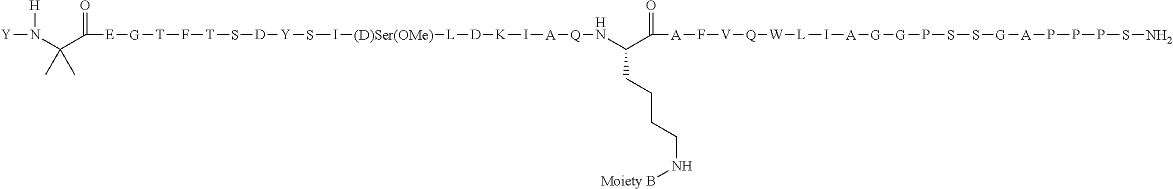 | Seq ID: 09 |
| 7 | 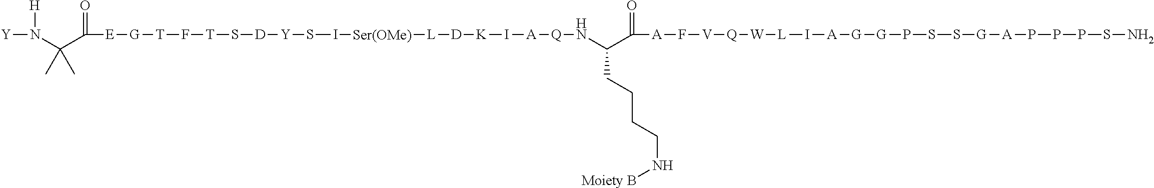 | Seq ID: 10 |
| 8 | 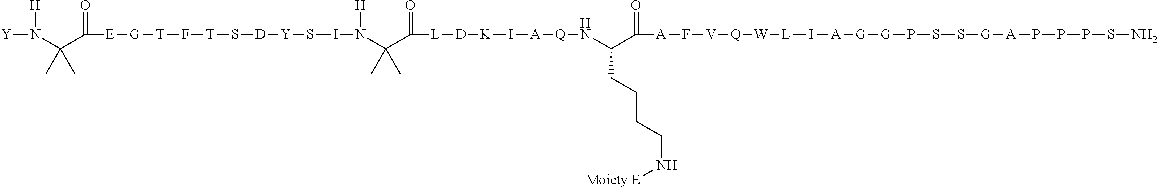 | Seq ID: 05 |
| 9 | 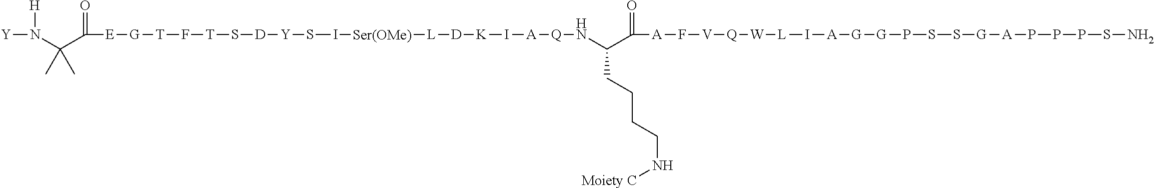 | Seq ID: 10 |
| 10 | 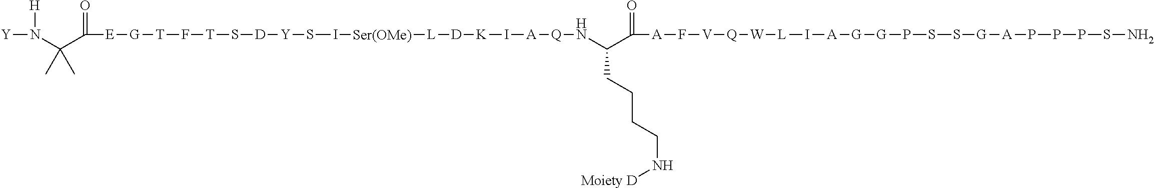 | Seq ID: 10 |
| 11 | 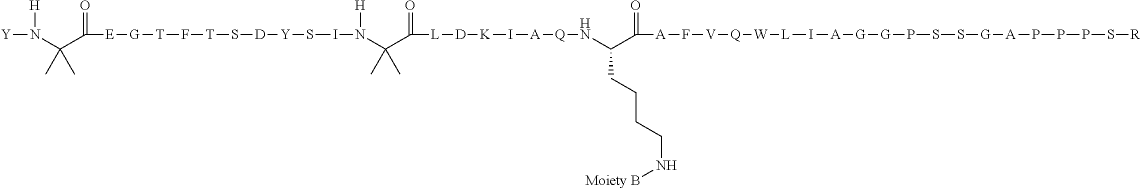 | Seq ID: 11 |
| 12 | 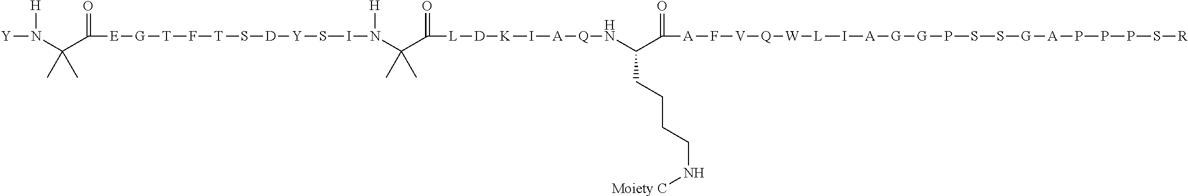 | Seq ID: 11 |
| 13 | 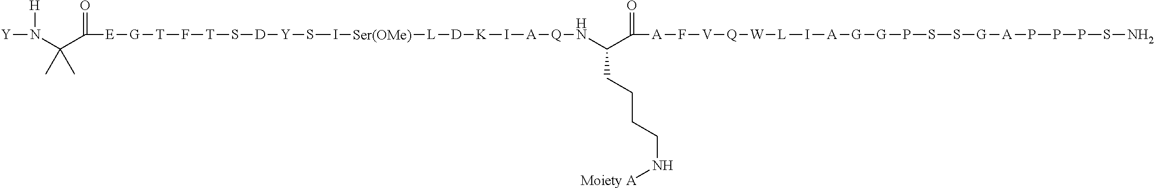 | Seq ID: 10 |
| 14 | 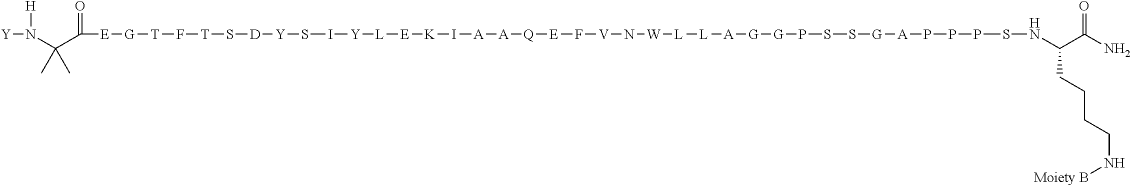 | Seq. ID: 12 |
| 15 | 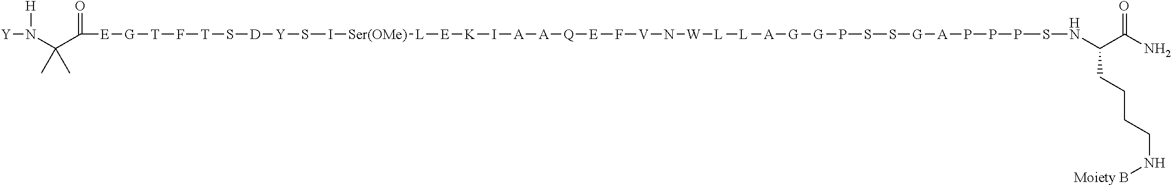 | Seq. ID: 13 |
| 16 | 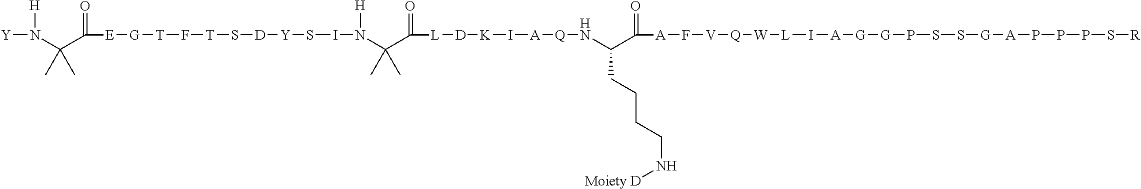 | Seq ID: 11 |

*Unless stated otherwise all the amino acids mentioned are in “L” configuration.

wherein, the structures of Moieties A, B, C & D are disclosed in Table 2.

TABLE 2
Structure of Moiety A, B, C, D and E

| | |
|---|---|
| Moiety A | 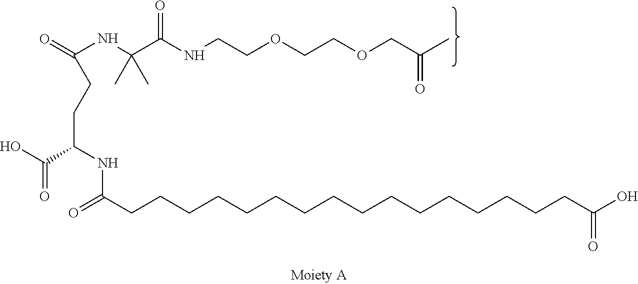 [SMILES: CC(=O)COCCOCCNC(=O)C(C)(C)NC(=O)CC[C@H](NC(=O)CCCCCCCCCCCCCCCCC(=O)O)C(=O)O] |
| Moiety B | 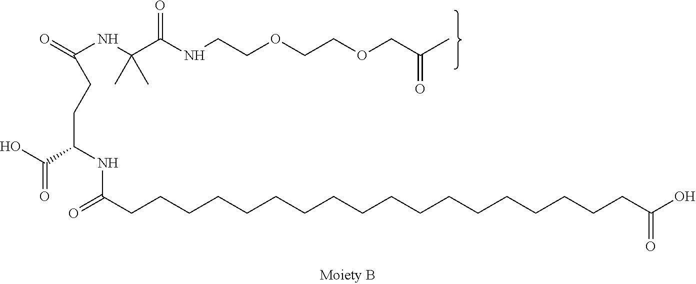 [SMILES: CC(=O)COCCOCCNC(=O)C(C)(C)NC(=O)CC[C@H](NC(=O)CCCCCCCCCCCCCCCCCCC(=O)O)C(=O)O] |
| Moiety C | 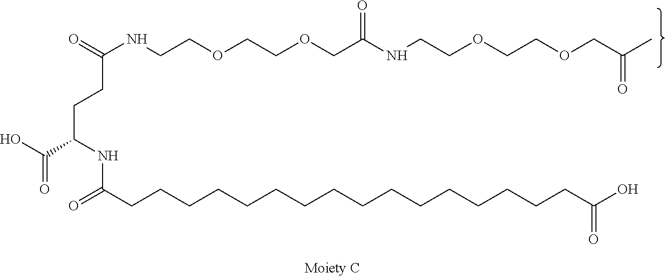 [SMILES: CC(=O)COCCOCCNC(=O)COCCOCCNC(=O)CC[C@H](NC(=O)CCCCCCCCCCCCCCCCC(=O)O)C(=O)O] |
| Moiety-D | 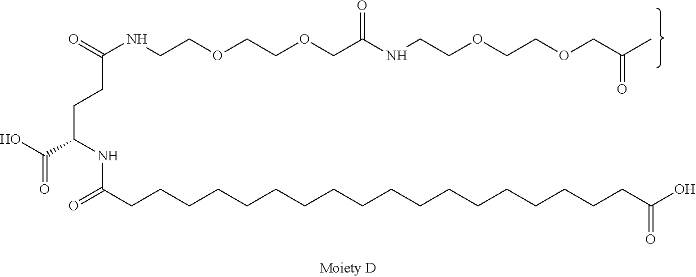 [SMILES: CC(=O)COCCOCCNC(=O)COCCOCCNC(=O)CC[C@H](NC(=O)CCCCCCCCCCCCCCCCCCC(=O)O)C(=O)O] |
| Moiety E | 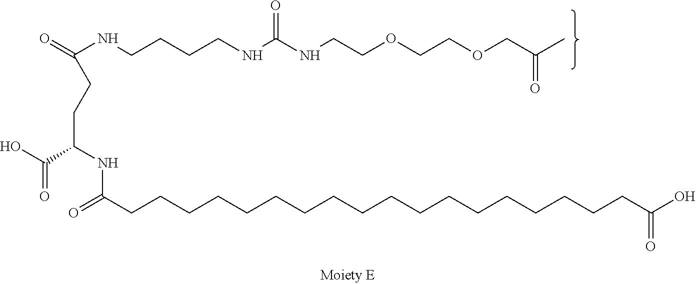 [SMILES: CC(=O)COCCOCCNC(=O)NCCCCNC(=O)CC[C@H](NC(=O)CCCCCCCCCCCCCCCCCCC(=O)O)C(=O)O] |

In another aspect, the present invention provides a method of treating or preventing hyperglycemia, type 2 diabetes, impaired glucose tolerance, type 1 diabetes, obesity, hypertension, hyperlipidemia, syndrome X, dyslipidemia, cognitive disorders, atherosclerosis, myocardial infarction, coronary heart disease, stroke, inflammatory bowel syndrome, dyspepsia, alcoholism and gastric ulcers in a patient, comprising administering to a patient in need thereof, an effective amount of a polypeptide of the present invention or pharmaceutically acceptable salt thereof.

In another aspect, invention provides a method of treatment of type 2 diabetes in a patient comprising administering to a patient in need of such treatment an effective amount of a polypeptide of the present invention or a pharmaceutically acceptable salt thereof.

In another aspect, invention provides a method of treatment of obesity in a patient comprising administering to a patient in need of such treatment an effective amount of a polypeptide of the present invention or a pharmaceutically acceptable salt thereof.

In another aspect, invention provides a method of treatment of hyperlipidemia in a patient comprising administering to a patient in need of such treatment an effective amount of a polypeptide of the present invention or a pharmaceutically acceptable salt thereof.

The term “effective amount or amount effective” as used herein refers to an amount of the polypeptide which is sufficient, upon single or multiple dose administration(s) to a subject, in curing, alleviating, relieving or partially addressing the clinical manifestation of given disease or state and its complications beyond that expected in the absence of such treatment. Thus, the result can be a reduction and/or alleviation of the signs, symptoms, or causes of a disease, or any other desired alteration of a biological system. It is understood that “a therapeutically effective amount” can vary from subject to subject depending on age, weight, general condition of the subject, the condition being treated, the severity of the condition being treated, and the judgment of the prescribing physician.

In another aspect, the present invention provides a pharmaceutical composition comprising a polypeptide of the present invention or pharmaceutically acceptable salt thereof with one or more of a pharmaceutically acceptable carrier, diluent, or excipient.

The polypeptides of the present invention or pharmaceutically acceptable salts thereof are preferably formulated as pharmaceutical compositions administered by parenteral routes (e.g., subcutaneous, intravenous, intraperitoneal, intramuscular, or transdermal). Such pharmaceutical compositions and processes for preparing same are well known in the art. (See, e.g., Remington: The Science and 50 Practice of Pharmacy (D. B. Troy, Editor, 21st Edition, Lippincott, Williams & Wilkins, 2006).

In another aspect, the present invention provides a polypeptide of the present invention or pharmaceutically acceptable salt thereof or a pharmaceutical composition comprising a polypeptide of the present invention or pharmaceutically acceptable salt thereof, for use in the treatment or prevention of a disease in a patient, wherein said disease is selected from the group consisting of hyperglycemia, type 2 diabetes, impaired glucose tolerance, type 1 diabetes, obesity, hypertension, hyperlipidemia, syndrome X, dyslipidemia, cognitive disorders, atherosclerosis, myocardial infarction, coronary heart disease, stroke, inflammatory bowel syndrome, dyspepsia, alcoholism and gastric ulcers.

In some embodiments, the polypeptide or pharmaceutically acceptable salt thereof or a pharmaceutical composition is provided simultaneously, separately, or sequentially in combination with an effective amount of one or more additional therapeutic agents.

The present invention may involve one or more embodiments. It is to be understood that the embodiments below are illustrative of the present invention and are not intended to limit the claims to the specific embodiments exemplified. It is also to be understood that the embodiments defined herein may be used independently or in conjunction with any definition, any other embodiment defined herein. Thus, the invention contemplates all possible combinations and permutations of the various independently described embodiments.

### EXAMPLES

Instruments and analytical methods: Instruments used for characterization and analysis of the compounds of the present invention are HPLC (Waters e2695 Alliance; Detector Waters (2489 UV/Visible)).
Mass instrument: HPLC: Waters e2695 Alliance; Detector: Acquity—QDa.
The final compounds of the present disclosure were purified by preparative HPLC procedure as outlined below:
Preparative HPLC: WATERS 2555 Quaternary gradient module (Max Total Flow: 300 mL/min, Max Pressure: 3000 psi) or Shimadzu LC-8A (Max Total Flow: 150 mL, Max Pressure: 30 Mpa), Column: Phenyl, 10μ Flow: 75 mL/min

### Mobile Phase:

| | For first | For second | For third |
|---|---|---|---|
| | purification | purification | purification |
| Mobile | pH 8.0 Phosphate | 1% Acetic acid in | pH 8.2 Ammonium |
| Phase A | buffer | water | formate buffer |
| Mobile | Acetonitrile | 1% Acetic acid in | Acetonitrile |
| Phase B | | Acetonitrile:n- | |
| | | Propanol (50:50) | |
| Gradient | 15 to 45% Mobile | 20 to 50% Mobile | 20 to 50% Mobile |
| | Phase-B in 300 | Phase-B in 250 min | Phase-B in 250 min |
| | min | | |

The purity of the compounds of the present disclosure was analyzed by RP-HPLC method as outlined below:

### HPLC Method B1:

Column: YMC Pack-Phenyl (4.6 mm×150 mm 3μ)
Eluent: Mobile Phase A: 0.1% Trifluroacetic acid in Water
Mobile phase B: 0.1% Trifluroacetic acid in Acetonitrile
Flow rate: 1.5 mL/min
Detection: UV detection at 210 nm

### Column Temperature: 50° C.

### Run Time: 50 min.

### Gradient:

| Time | Mobile Phase A % | Mobile Phase B % |
|---|---|---|
| 0.01 | 90 | 10 |
| 35.0 | 20 | 80 |
| 40.0 | 20 | 80 |
| 41.0 | 90 | 10 |
| 50.0 | 90 | 10 |

### HPLC Method B2:

Column: Xbridge Peptide BEH C18 (4.6 mm×250 mm, 3.5μ)

### Eluent: Mobile Phase A: Buffer: Acetonitrile (900:100)

Mobile phase B: Buffer: Acetonitrile (300:700)
Buffer: Potassium dihydrogen orthophosphate in water, pH adjusted to 3.0±0.1 with orthophosphoric acid
Flow rate: 1.0 mL/min
Detection: UV detection at 210 nm

### Column Temperature: 65° C.

Sample Tray temperature: 5° C.

### Run Time: 40 min.

| Time | Mobile Phase A % | Mobile Phase B % |
|---|---|---|
| 0 | 55 | 45 |
| 5 | 41 | 59 |
| 35 | 40 | 60 |
| 35.1 | 55 | 45 |
| 40 | 55 | 45 |

### Method B3:

Column: Xbridge Peptide BEH C18 (4.6 mm×250 mm, 3.5μ)

### Eluent: Mobile Phase A: Buffer: Acetonitrile (900:100)

Mobile phase B: Buffer: Acetonitrile (300:700)
Buffer: Potassium dihydrogen orthophosphate in water, pH adjusted to 3.0±0.1 with orthophosphoric acid
Flow rate: 1.0 mL/min
Detection: UV detection at 210 nm

### Column Temperature: 65° C.

Sample Tray temperature: 5° C.

### Run Time: 65 min.

| Time | Mobile Phase A % | Mobile Phase B % |
|---|---|---|
| 0 | 55 | 45 |
| 5 | 40 | 60 |
| 60 | 35 | 65 |
| 60.1 | 55 | 45 |
| 65 | 55 | 45 |

### Method B4:

Column: Xbridge Peptide BEH C18 (4.6 mm×250 mm, 3.5μ)

### Eluent: Mobile Phase A: Buffer: Acetonitrile (900:100)

Mobile phase B: Buffer: Acetonitrile (300:700)
Buffer: Potassium dihydrogen orthophosphate in water, pH adjusted to 3.0±0.1 with orthophosphoric acid
Flow rate: 0.8 mL/min
Detection: UV detection at 210 nm

### Column Temperature: 65° C.

Sample Tray temperature: 5° C.

### Run Time: 90 min.

| Time | Mobile Phase A % | Mobile Phase B % |
|---|---|---|
| 0 | 55 | 45 |
| 3 | 55 | 45 |
| 5 | 40 | 60 |
| 60 | 39 | 61 |
| 65 | 0 | 100 |
| 75 | 0 | 100 |
| 75.01 | 55 | 45 |
| 90 | 55 | 45 |

### Method B5:

Column: Xbridge Peptide BEH C18 (4.6 mm×250 mm, 3.5μ)

### Eluent: Mobile Phase A: Buffer: Acetonitrile (900:100)

Mobile phase B: Buffer: Acetonitrile (300:700)
Buffer: Potassium dihydrogen orthophosphate in water, pH adjusted to 3.0±0.1 with orthophosphoric acid
Flow rate: 1.0 mL/min
Detection: UV detection at 210 nm

### Column Temperature: 65° C.

Sample Tray temperature: 10° C.

### Run Time: 60 min.

| Time | Mobile Phase A % | Mobile Phase B % |
|---|---|---|
| 0 | 55 | 45 |
| 2 | 41 | 59 |
| 50 | 40 | 60 |
| 51 | 55 | 45 |
| 60 | 55 | 45 |

### Method of Preparation

### Example 1. Preparation of 2-[2-[2-[[2-[[(4S)-5-tert-butoxy-4-[(18-tert-butoxy-18-oxo-octadecanoyl)amino]-5-oxo-pentanoyl]amino]-2-methyl-propanoyl]amino]ethoxy]ethoxy]acetic Acid (Moiety A-di-tert-butyl Ester)

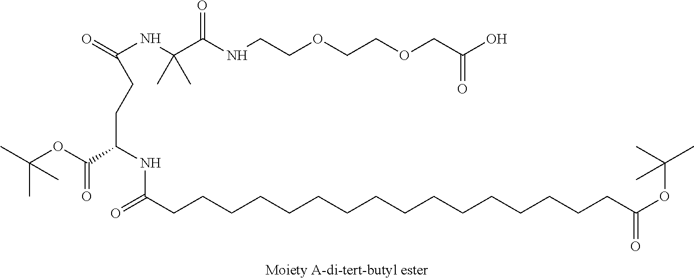 [SMILES: CC(C)(C)OC(=O)CCCCCCCCCCCCCCCCC(=O)N[C@@H](CCC(=O)NC(C)(C)C(=O)NCCOCCOCC(=O)O)C(=O)OC(C)(C)C]

Moiety A-di-tert-butyl ester was prepared using solid phase synthesis using 2-chlorotrityl chloride resin. 2-[2-(2-Fmoc-aminoethoxy)ethoxy]acetic acid was attached to 2-chlorotrityl chloride resin in presence of DIPEA to yield 2-[2-(2-Fmoc-aminoethoxy)ethoxy]acetic acid-2-Cl-Trt-Resin. The Fmoc protecting group was removed by selective de-blocking of amino group using piperidine followed by coupling with Fmoc-Aib-OH in THF: DMAc/THF using DIPC and HOBt which yielded 2-[2-[2-[(2-Fmoc-amino-2-methyl-propanoyl)amino]ethoxy]ethoxy]acetic acid-2-Cl-Trt-Resin. The Fmoc group was removed by selective de-blocking using piperidine and the free amino group was coupled with Fmoc-Glu-OtBu using HOBt and DIPC to yield 2-[2-[2-[[2-[[(4S)-4-Fmoc-amino-5-tert-butoxy-5-oxo-pentanoyl]amino]-2-methyl-propanoyl]amino]ethoxy]ethoxy]acetic acid-2-Cl-Trt-Resin. The Fmoc group of the resultant compound was selectively de-blocked using piperidine and the free amino group was then coupled with octadecanedioic acid mono tert butyl ester to give 2-[2-[2-[[2-[[(4S)-5-tert-butoxy-4-[(18-tert-butoxy-18-oxo-octadecanoyl)amino]-5-oxo-pentanoyl]amino]-2-methyl-propanoyl]-amino]ethoxy]ethoxy]acetic acid-2-Cl-Trt-Resin. The intermediate was then cleaved from 2-Cl-Trt-Resin using trifluoroethanol:DCM (1:1) to obtain the title compound (Moiety A-di-tert-butyl ester). (LCMS=m/z: 786.39 (M+H⁺)).

### Example 2. Preparation of 2-[2-[2-[[2-[[(4S)-5-tert-butoxy-4-[(20-tert-butoxy-20-oxo-icosanoyl)amino]-5-oxo-pentanoyl]amino]-2-methyl-propanoyl]amino]ethoxy]ethoxy]acetic Acid (Moiety B-di-tert-butyl Ester)

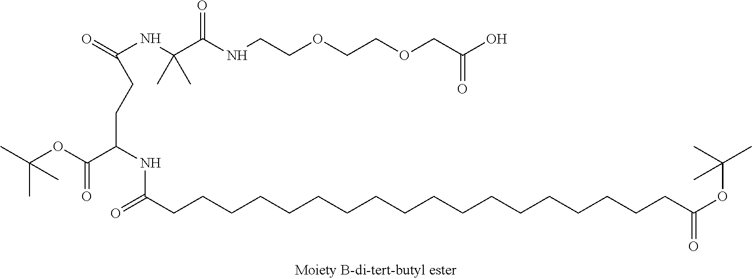 [SMILES: CC(C)(C)OC(=O)CCCCCCCCCCCCCCCCCCC(=O)NC(CCC(=O)NC(C)(C)C(=O)NCCOCCOCC(=O)O)C(=O)OC(C)(C)C]

2-[2-[2-[[2-[[(4S)-4-Fmoc-amino-5-tert-butoxy-5-oxo-pentanoyl]amino]-2-methyl-propanoyl]amino]ethoxy]ethoxy]acetic acid-2-Cl-Trt-Resin was prepared as described in Example 1 and was subjected to selective de-protection using piperidine and the free amino group was then coupled with 20-(tert-butoxy)-20-oxoicosanoic acid to give 2-[2-[2-[[2-[[(4S)-5-tert-butoxy-4-[(20-tert-butoxy-20-oxo-icosanoyl)amino]-5-oxo-pentanoyl]amino]-2-methyl-propanoyl]amino]ethoxy]ethoxy]acetic acid-2-Cl-Trt-Resin. The intermediate was then cleaved from 2-Cl-Trt-Resin using trifluoroethanol:DCM (1:1) to obtain the tile compound (Moiety B-di-tert-butyl ester). (LCMS=m/z: 814.10 (M+H⁺)).

### Example 3: Preparation of 2-[2-[2-[[2-[2-[2-[[5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic Acid (Moiety C-di-tert-butyl Ester)

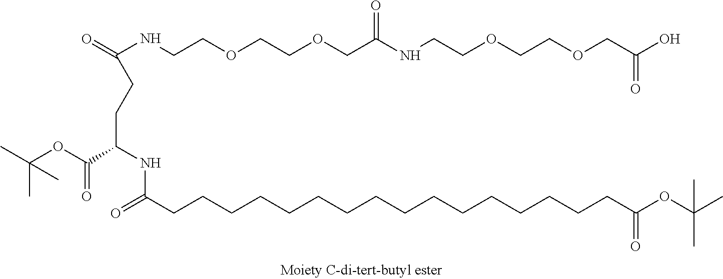 [SMILES: CC(C)(C)OC(=O)CCCCCCCCCCCCCCCCC(=O)N[C@@H](CCC(=O)NCCOCCOCC(=O)NCCOCCOCC(=O)O)C(=O)OC(C)(C)C]

Moiety C-di-tert-butyl ester was prepared using solid phase synthesis using 2-chlorotrityl chloride resin. 2-[2-(2-Fmoc-aminoethoxy)ethoxy]acetic acid was attached to 2-chlorotrityl chloride resin in presence of DIPEA to yield 2-[2-(2-Fmoc-aminoethoxy)ethoxy]acetic acid-2-Cl-Trt-Resin. The Fmoc protecting group was removed by selective de-blocking of amino group using piperidine followed by coupling with 2-[2-(2-Fmoc-aminoethoxy)ethoxy]acetic acid in THF using DIPC and HOBt which yielded {(Fmoc-amino-ethoxy)-ethoxy}-acetyl-{(-amino-ethoxy)-ethoxy}-acetic acid-2-Cl-Trt-Resin The Fmoc group was removed by selective de-blocking using piperidine and the free amino group was coupled with Fmoc-Glu-OtBu using HOBt and DIPC to yield Fmoc-Glu({(amino-ethoxy)-ethoxy}-acetyl-{(-amino-ethoxy)-ethoxy}-acetic acid-2-Cl-Trt-Resin)-OtBu The Fmoc group of the resultant compound was selectively de-blocked using piperidine and the free amino group was then coupled with octadecanedioic acid mono tert butyl ester to give 2-[2-[2-[[2-[2-[2-[[5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid-2-Cl-Trt-Resin. The intermediate was then cleaved from 2-Cl-Trt-Resin using trifluoroethanol:DCM (1:1) to obtain 2-[2-[2-[[2-[2-[2-[[5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid (Moiety C-di-tert-butyl ester) (LCMS=m/z: 846.10 (M+H⁺)).

### Example 4: Preparation of 2-[2-[2-[[2-[2-[2-[[5-tert-butoxy-4-[(20-tert-butoxy-20-oxo-icosanoyl)amino]-5-oxo-pentanoyl]amino]ethoxy]ethoxy]acetyl]amino]ethoxy]ethoxy]acetic Acid. (Moiety D-di-tert-butyl Ester)

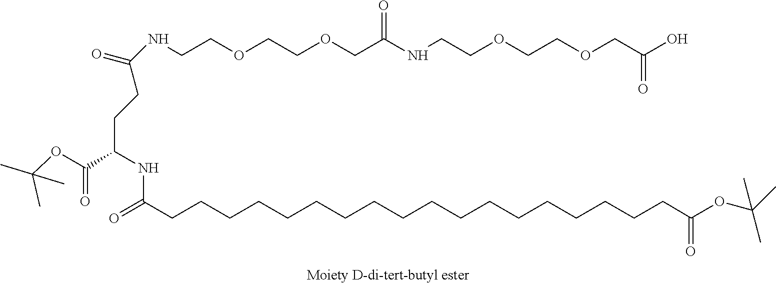 [SMILES: CC(C)(C)OC(=O)CCCCCCCCCCCCCCCCCCC(=O)N[C@@H](CCC(=O)NCCOCCOCC(=O)NCCOCCOCC(=O)O)C(=O)OC(C)(C)C]

Moiety D-di-tert-butyl ester was prepared using solid phase synthesis using 2-chlorotrityl chloride resin as schematically represented below. 2-[2-(2-Fmoc-aminoethoxy)ethoxy]acetic acid was attached to 2-chlorotrityl chloride resin in presence of DIPEA to yield 2-[2-(2-Fmoc-aminoethoxy)ethoxy]acetic acid-2-Cl-Trt-Resin. The Fmoc protecting group was removed by selective de-blocking of amino group using piperidine followed by coupling with 2-[2-(2-Fmoc-aminoethoxy)ethoxy]acetic acid in THF using DIPC and HOBt which yielded {(Fmoc-amino-ethoxy)-ethoxy}-acetyl-{(-amino-ethoxy)-ethoxy}-acetic acid-2-Cl-Trt-Resin The Fmoc group was removed by selective de-blocking using piperidine and the free amino group was coupled with Fmoc-Glu-OtBu using HOBt and DIPC to yield Fmoc-Glu({(amino-ethoxy)-ethoxy}-acetyl-{(-amino-ethoxy)-ethoxy}-acetic acid-2-Cl-Trt-Resin)-OtBu The Fmoc group of the resultant compound was selectively de-blocked using piperidine and the free amino group was then coupled with 20-(tert-Butoxy)-20-oxoicosanoic acid to give

2-[2-[2-[[2-[2-[2-[[5-tert-butoxy-4-[(20-tert-butoxy-20-oxo-icosanoyl)amino]-5-oxo-pentanoyl]amino]ethoxy]ethoxy]acetyl]amino]ethoxy]ethoxy]acetic acid-2-Cl-Trt-Resin. The intermediate was then cleaved from 2-Cl-Trt-Resin using trifluoroethanol:DCM (1:1) to obtain

2-[2-[2-[[2-[2-[2-[[5-tert-butoxy-4-[(20-tert-butoxy-20-oxo-icosanoyl)amino]-5-oxo-pentanoyl]amino]ethoxy]ethoxy]acetyl]amino]ethoxy]ethoxy]acetic acid (Moiety D-di-tert-butyl ester) (LCMS=m/z: 874.15 (M+H⁺)).

### Example 5: Preparation of 2-[2-[2-[4-[[5-tert-butoxy-4-[(20-tert-butoxy-20-oxo-icosanoyl)amino]-5-oxo-pentanoyl]amino]butylcarbamoylamino]ethoxy]ethoxy]acetic Acid (Moiety E-di-tert-butyl Ester)

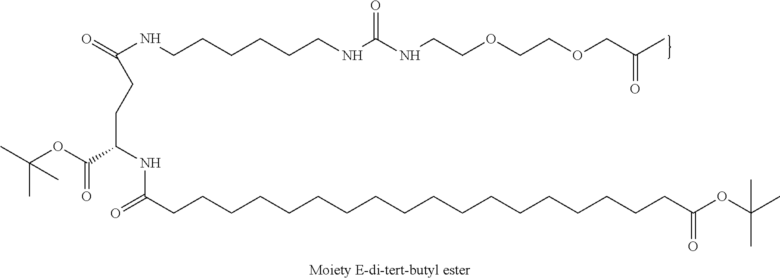 [SMILES: CC(=O)COCCOCCNC(=O)NCCCCCCNC(=O)CC[C@H](NC(=O)CCCCCCCCCCCCCCCCCCC(=O)OC(C)(C)C)C(=O)OC(C)(C)C]

Moiety E-di-tert-butyl ester was prepared using solid phase synthesis using 2-chlorotrityl chloride resin. 2-[2-(2-Fmoc-aminoethoxy)ethoxy]acetic acid was attached to 2-chlorotrityl chloride resin in presence of N,N′-di-isopropylethylamine (DIPEA) to yield 2-[2-(2-Fmoc-aminoethoxy)ethoxy]acetic acid-2-Cl-Trt-Resin. The Fmoc protecting group was removed by selective de-blocking of amino group using piperidine and the free amino group was then activated using p-nitrophenylchloroformate in THF and DIPEA followed by reaction with Fmoc-amino butylamine hydrochloride salt in THF: DMAc in presence of DIPEA which yielded 2-[2-[2-(4-Fmoc-aminobutylcarbamoylamino)ethoxy]ethoxy]acetic acid-2-Cl-Trt-Resin. The Fmoc group was removed by selective de-blocking using piperidine and the free amino group was then coupled to Fmoc-Glu-OtBu using 1-hydroxybenztriazole (HOBt) and N,N′-di-isopropylcarbodiimide (DIPC) which yielded 2-[2-[2-[4-[[(4S)-4-Fmoc-amino-5-tert-butoxy-5-oxo-pentanoyl]amino]butylcarbamoylamino]ethoxy]ethoxy]acetic acid-2-Cl-Trt-Resin which was selectively deblocked using piperidine and then coupled with 20-(tert-Butoxy)-20-oxoicosanoic acid to give intermediate 2-[2-[2-[4-[[5-tert-butoxy-4-[(20-tert-butoxy-20-oxo-icosanoyl)amino]-5-oxo-pentanoyl]amino]butylcarbamoylamino]ethoxy]ethoxy]acetic acid-2-Cl-Trt-Resin. The intermediate was then cleaved from 2-Cl-Trt-Resin using trifluoroethanol:DCM (1:1) to obtain 2-[2-[2-[4-[[5-tert-butoxy-4-[(20-tert-butoxy-20-oxo-icosanoyl)amino]-5-oxo-pentanoyl]amino]butylcarbamoylamino]ethoxy]ethoxy]acetic acid (LCMS=m/z: 843.14 (M+H⁺)). (Moiety E-di-tert-butyl ester).

### Example 6: Preparation of Compound 1

The parent peptide was synthesized by solid-phase method. The starting resin used for synthesis was Fmoc-Rink amide resin. Selectively de-blocking of Fmoc protected amino group of rink amide resin using piperidine followed by coupling of Fmoc-Ser(tBu)—OH with the Rink amide resin. The coupling was performed by using DIPC-HOBt to yield Fmoc-Ser(tBu)-Rink amide Resin, this complete one cycle. Acetic anhydride and DIPEA/pyridine was used to terminate/cap the uncoupled amino groups at every amino acid coupling. Selective de-blocking of amino group of Fmoc-Ser(tBu)-Rink amide Resin using piperidine, then coupling with Fmoc-Pro-OH using HOBt and DIPC yielded Fmoc-Pro-Ser(tBu)-rink amide Resin. This completes 2^{nd} cycle. Acetic anhydride and DIPEA/pyridine was used to terminate the uncoupled amino groups at every amino acid coupling.

The above 3 steps, i.e., selective Capping, deblocking of Fmoc-protection of amino acid attached to the resin and coupling of next amino acid residue in sequence with Fmoc-protected amino group were repeated for remaining 36 amino acid residues and last coupling was done with Boc protected amino acids (i.e., Boc-Tyr (tBu)—OH). The selective deblocking, i.e., capping of uncoupled amino group done by using Acetic anhydride and DIPEA/pyridine, deprotection of Fmoc group was done using piperidine and coupling with next Fmoc and/or Boc protected amino acid was done using HOBt/DIPC. The side chain of the Fmoc/Boc-protected amino acids were protected orthogonally, e.g., hydroxyl group of Serine, Tyrosine or Threonine were protected with tert-butyl(-tBu) group, amino group of Lysine was protected with tert-butyloxycarbonyl (-Boc) and (4,4-dimethyl-2,6-dioxocyclohex-1-ylidene)-3-methylbutyl (IVDde) group respectively, carboxylic acid groups of aspartic acid or glutamic acid were protected with -tBu group and amide group of glutamine was protected with trityl (-Trt) group. The above mentioned three steps, i.e., selective capping, deblocking and then coupling with next Fmoc protected amino acids were performed and also Boc-Tyr(tBu)—OH is used at last to get Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val- Gln(Trt)-Trp-Leu-Ile-Ala-Gly-Gly-Pro-Ser(tBu)-Ser(tBu)-Gly-Ala-Pro-Pro-Pro-Ser(tBu)-Rink amide resin.

De-protection of IVDde group of peptide resin using hydrazine hydrate followed by coupling of Moiety A-di-tert butyl ester was performed by using DIPC-HOBt to yield protected Compound 1 resin.

Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(NH-Moiety A di-tert-butyl ester)-Ala-Phe-Val-Gln(Trt)-Trp-Leu-Ile-Ala-Gly-Gly-Pro-Ser(tBu)-Ser(tBu)-Gly-Ala-Pro-Pro-Pro-Ser(tBu)-Rink amide resin. Cleavage and de-protection using tri-fluoroacetic acid with ethane-1,2-dithiol and tri-isopropylsilane followed by purification through preparative HPLC resulted in Compound 1. The HPLC purity of Compound 1 was assessed by Method B2. Mass (LCMS): m/z=1182.41 (MH₄⁴⁺), Calculated Mass=4725.61; HPLC Purity: 97.77% (Method B2), RT=19.9 min.

### Example 7: Synthesis of Compound 2

Compound 2 was prepared by solid phase method as per the analogous process given for Example 6, except here Moiety B-di-tert butyl ester was coupled with Peptide resin, followed by cleavage, de protection and preparative purification using HPLC resulted in Compound 2. The HPLC purity of Compound 2 was assessed by Method B2.

Mass (LCMS): m/z=1189.36 (MH₄⁴⁺), Calculated Mass=4753.41; HPLC Purity: 94.50% (Method B2), RT=22.1 min.

### Example 8: Synthesis of Compound 3

The parent peptide was synthesized by solid-phase method. The starting resin used for synthesis was Fmoc-Rink amide resin. Selectively de-blocking of Fmoc protected amino group of rink amide resin using piperidine followed by coupling of Fmoc-Ser(tBu)-OH with the Rink amide resin. The coupling was performed by using DIPC-HOBt to yield Fmoc-Ser(tBu)-Rink amide Resin, this complete one cycle. Acetic anhydride and diisopropylethyl amine/pyridine was used to terminate/cap the uncoupled amino groups at every amino acid coupling. Selective de-blocking of amino group of Fmoc-Ser(tBu)-Rink amide Resin using piperidine, then coupling with Fmoc-Pro-OH using HOBt and DIPC yielded Fmoc-Pro-Ser(tBu)-rink amide Resin. This completes 2^{nd} cycle. Acetic anhydride and diisopropylethyl amine/pyridine was used to terminate the uncoupled amino groups at every amino acid coupling.

The above 3 steps, i.e., selective Capping, deblocking of Fmoc-protection of amino acid attached to the resin and coupling of next amino acid residue in sequence with Fmoc-protected amino group were repeated for remaining 37 amino acid residues. The selective deblocking, i.e. capping of uncoupled amino group done by using Acetic anhydride and diisopropylethylamine/pyridine, deprotection of Fmoc group was done using piperidine and coupling with next Fmoc protected amino acid was done using HOBt/DIPC. The side chain of the Fmoc-protected amino acids were protected orthogonally, e.g., hydroxyl group of serine was protected with tert-butyl(-tBu) group and O-Methyl (OMe) group, Tyrosine or Threonine were protected with tert-butyl(-tBu) group, amino group of Lysine was protected with tert-butyloxycarbonyl (-Boc) and (4,4-dimethyl-2,6-dioxocyclohex-1-ylidene)-3-methylbutyl (IVDde) group respectively, carboxylic acid groups of aspartic acid or glutamic acid were protected with -tBu group and amide group of glutamine was protected with trityl (-Trt) group. The above mentioned three steps, i.e., selective capping, deblocking and then coupling with next Fmoc protected amino acid were performed to get Fmoc-Tyr(tBu)-(D)Ser(OMe)-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly-Pro-Ser(tBu)-Ser(tBu)-Gly- Ala-Pro-Pro-Pro-Ser(tBu)-Rink amide resin.

De-blocking of Fmoc-Tyr(tBu)-(D)Ser(OMe)-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly-Pro- Ser(tBu)-Ser(tBu)-Gly-Ala-Pro-Pro-Pro-Ser(tBu)-Rink amide resin. using piperidine followed by Boc protection of Peptide resin using Boc anhydride to yield Boc-Tyr(tBu)-(D)Ser(OMe)-Glu(otBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe- Val-Gln(Trt)-Trp-Leu-Ile-Ala-Gly-Gly-Pro-Ser(tBu)-Ser(tBu)-Gly-Ala-Pro-Pro-Pro-Ser(tBu)-Rink amide resin. De-protection of IVDde group of peptide resin using Hydrazine hydrate followed by coupling of moiety B-di-tert butyl ester was performed by using diisopropylcarbodiimide, N-hydroxybenzotriazole (DIPC-HOBt) as coupling reagent in presence of which yielded Compound 3 resin.

Boc-Tyr(tBu)-(D)Ser(OMe)-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(NH-moiety B-di-tert butyl ester)-Ala-Phe-Val-Gln(Trt)-Trp-Leu-Ile-Ala-Gly-Gly-Pro-Ser(tBu)-Ser(tBu)-Gly-Ala-Pro-Pro-Pro-Ser(tBu)-Rink amide resin. Cleavage and de-protection using trifluoroacetic acid with ethane-1,2-dithiol and triisopropylsilane followed by purification through preparative HPLC resulted in Compound 3. The HPLC purity of Compound 3 was assessed by Method B2.

Mass (LCMS): m/z=1193.70 (MH₄⁴⁺), Calculated Mass=4770.77; HPLC Purity: 91.96% (Method B2), RT=29.0 min.

### Example 9: Synthesis of Compound 4

Compound 4 was prepared by solid phase method as per the analogous process given for Example 8, wherein for Compound 4 Fmoc-Ser(OMe)—OH was used at position 2 instead of Fmoc-D-Ser(OMe)—OH to get Boc-Tyr(tBu)-Ser(OMe)-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Tr)-Lys(IVDde)-Ala-Phe-Val-Gln(Trt)-Trp-Leu-Ile-Ala-Gly- Gly-Pro-Ser(tBu)-Ser(tBu)-Gly-Ala-Pro-Pro-Pro-Ser(tBu)-Rink amide resin. Then coupling with Moiety B-di-tert butyl ester followed by cleavage, de protection and preparative purification using HPLC resulted in Compound 4. The HPLC purity of Compound 4 was assessed by Method B2.

Mass (LCMS): m/z=1193.68 (MH₄⁴⁺), Calculated Mass=4770.69; HPLC Purity: 95.52% (Method B2), RT=26.2 min.

### Example 10: Synthesis of Compound 5

Compound 5 was prepared by solid phase method as per the analogous process given for Example 8, wherein for Compound 5 Fmoc-(D)-Tyr(OEt)-OH was used at position 1 instead of Fmoc-Tyr(tBu)—OH and Fmoc-Aib-OH was used at position 2^{nd} instead of Fmoc-D-Ser(OMe)-OH to get Boc-(D)-Tyr(OEt)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly-Pro-Ser(tBu)-Ser(tBu)- Gly-Ala-Pro-Pro-Pro-Ser(tBu)-Rink amide resin.

Then coupling with Moiety B-di-tert butyl ester followed by cleavage, de protection and preparative purification using HPLC resulted in Compound 5. The HPLC purity of Compound 5 was assessed by Method B3.

Mass (LCMS): m/z=1196.34 (MH₄⁴⁺), Calculated Mass=4781.33; HPLC Purity: 93.86% (Method B3), RT=38.8 min.

### Example 11: Synthesis of Compound 6

Compound 6 was prepared by solid phase method as per the analogous process given for Example 8, wherein for Compound 6 Fmoc-(D)Ser (OMe)—OH was used at position 13 instead of Fmoc-Aib-OH and Fmoc-Aib-OH was used at position 2^{nd} instead of Fmoc-D-Ser(OMe)—OH to get Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-(D)Ser(OMe)-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly-Pro-Ser(tBu)- Ser(tBu)-Gly-Ala-Pro-Pro-Pro-Ser(tBu)-Rink amide resin.

Then coupling with Moiety B-di-tert butyl ester followed by cleavage, de protection and preparative purification using HPLC resulted in Compound 6. The HPLC purity of Compound 6 was assessed by Method B2.

Mass (LCMS): m/z=1191.03 (MH₄⁴⁻), Calculated Mass: 4768.15; HPLC Purity: 94.74% (Method B2), RT=27.1 min.

### Example 12: Synthesis of Compound 7

Compound 7 was prepared by solid phase method as per the analogous process given for Example 8, wherein for Compound 7 Fmoc-Ser(OMe)—OH was used at position 13 instead of Fmoc-Aib-OH and Fmoc-Aib-OH was used at position 2nd instead of Fmoc-D-Ser(OMe)—OH to get Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Ser(OMe)-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly-Pro-Ser(tBu)- Ser(tBu)-Gly-Ala-Pro-Pro-Pro-Ser(tBu)-Rink amide resin.

Then coupling with Moiety B-di-tert butyl ester followed by cleavage, de protection and preparative purification using HPLC resulted in Compound 7. The HPLC purity of Compound 7 was assessed by Method B2.

Mass (LCMS): m/z=1193.67 (MH₄⁴⁺), Calculated Mass=4770.65; HPLC Purity: 95.4% (Method B2), RT=26.4 min.

### Example 13: Synthesis of Compound 8

Compound 8 was prepared by solid phase method as per the analogous process given for Example 8, wherein for Compound 8 Fmoc-Aib-OH was used at position 2nd instead of Fmoc-D-Ser(OMe)—OH to get Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly-Pro- Ser(tBu)-Ser(tBu)-Gly-Ala-Pro-Pro-Pro-Ser(tBu)-Rink amide resin.

Then coupling with Moiety E-di-tert butyl ester followed by cleavage, de protection and preparative purification using HPLC resulted in Compound purification using HPLC resulted in Compound 8. The HPLC purity of Compound 8 was assessed by Method B4.

Mass (LCMS): m/z=1196.55 (MH₄⁴⁺), Calculated Mass=4782.168; HPLC Purity: 97.37% (Method B4), RT=25.6 min.

### Example 14: Synthesis of Compound 9

Compound 9 was prepared by solid phase method as per the analogous process given for Example 8, wherein for Compound 9 Fmoc-Ser(OMe)—OH was used at position 13 instead of Fmoc-Aib-OH and Fmoc-Aib-OH was used at position 2nd instead of Fmoc-D-Ser(OMe)—OH to get Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Ser(OMe)-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly-Pro-Ser(tBu)- Ser(tBu)-Gly-Ala-Pro-Pro-Pro-Ser(tBu)-Rink amide resin.

Then coupling with Moiety C-di-tert butyl ester followed by cleavage, de protection and preparative purification using HPLC resulted in Compound 9. The HPLC purity of Compound 9 was assessed by Method B2.

Mass (LCMS): m/z=1201.7 (MH₄⁴⁺), Calculated Mass=4802.8; HPLC Purity: 97.30% (Method B2), RT=15.3 min.

### Example 15: Synthesis of Compound 10

Compound 10 was prepared by solid phase method as per the analogous process given for Example 8, wherein for Compound 10 Fmoc-Ser (OMe)—OH was used at position 13 instead of Fmoc-Aib-OH and Fmoc-Aib-OH was used at position 2^{nd} instead of Fmoc-D-Ser(OMe)—OH to get Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Ser(OMe)-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly-Pro-Ser(tBu)- Ser(tBu)-Gly-Ala-Pro-Pro-Pro-Ser(tBu)-Rink amide resin.

Then coupling with Moiety D-di-tert butyl ester followed by cleavage, de protection and preparative purification using HPLC resulted in Compound 10. The HPLC purity of Compound 10 was assessed by Method B2.

Mass (LCMS): m/z=1610.78 (MH₃³⁺), Calculated Mass=4829.316; HPLC Purity: 93.41% (Method B2), RT=20.3 min.

### Example 16: Synthesis of Compound 11

The parent peptide was synthesized by solid-phase method. The starting resin used for synthesis was Wang resin. Fmoc protected Arg(pbf) was used for coupling with the Wang resin. The coupling was performed by using diisopropylcarbodiimide, N-hydroxybenzotriazole (DIC-HOBt) as coupling reagent in presence of 4-dimethylaminopyridine (DMAP) which yielded Fmoc-Arg(pbf)-Wang Resin. Selective de-blocking of amino group of Fmoc-Arg(pbf)-Wang Resin using piperidine followed by coupling with Fmoc-Ser(tBu)—OH using HOBt/DIPC yielded Fmoc-Ser(tBu)-Arg(pbf)-Wang Resin. This completes one cycle. Acetic anhydride and diisopropylethyl amine/pyridine was used to terminate the uncoupled amino groups at every amino acid coupling.

The above 3 steps i.e., selective de-blocking of Fmoc-protection of amino acid attached to the resin, coupling of next amino acid residue in sequence with Fmoc-protected amino group and capping were repeated for remaining 38 amino acid residues, The side chain of the Fmoc-protected amino acids were protected orthogonally, e.g., hydroxyl group of Serine, Tyrosine or Threonine were protected with tert-butyl(-tBu) group, amino group of Lysine was protected with tert-butyloxycarbonyl (-Boc) and (4,4-dimethyl-2,6-dioxocyclohex-1-ylidene)-3-methylbutyl (IVDde) group respectively and carboxylic acid groups of aspartic acid or glutamic acid were protected with -tBu group, amide group of glutamine was protected with trityl (-Trt) group and Side chain of arginine protected with pbf group. The above mentioned three steps, i.e., selective capping, deblocking and then coupling with next Fmoc protected amino acid were performed to get Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val- Gln(Trt)-Trp-Leu-Ile-Ala-Gly-Gly-Pro-Ser(tBu)-Ser(tBu)-Gly-Ala-Pro-Pro-Pro-Ser(tBu)-Arg(pbf)-Wang resin.

De-blocking of Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val-Gln(Trt)-Trp-Leu-Ile-Ala-Gly-Gly-Pro-Ser(tBu)- Ser(tBu)-Gly-Ala-Pro-Pro-Pro-Ser(tBu)-Arg(pbf)-Wang resin, using piperidine followed by Boc protection of Peptide resin using Boc anhydride to yield Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Aib-Leu-Asp(OtBu)-Lys(Boc)-Ile-Ala-Gln(Trt)-Lys(IVDde)-Ala-Phe-Val- Gln(Trt)-Trp-Leu-Ile-Ala-Gly-Gly-Pro-Ser(tBu)-Ser(tBu)-Gly-Ala-Pro-Pro-Pro-Ser(tBu)-Arg(pbf)-Wang resin. De-protection of IVDde group of peptide resin was done using hydrazine hydrate and then it was coupled with Moiety B-di-tert butyl ester using diisopropylcarbodiimide, N-hydroxybenzotriazole (DIPC-HOBt) as coupling reagent to yield intermediate protected Compound 11 resin. Cleavage and de-protection from resin using trifluoroacetic acid with ethane-1,2-dithiol, triisopropylsilane followed by purification through preparative HPLC resulted in Compound 11.

The HPLC purity of Compound 11 was assessed by Method B2.

Mass (LCMS): m/z=1228.8 (MH₄⁴⁺), Calculated Mass=4911.17; HPLC Purity: 98.22% (Method B2), RT=23.3 min.

### Example 17: Synthesis of Compound 12

Compound 12 was prepared by solid phase method as per the analogous process given for Example 16 except here Moiety C-di-tert butyl ester was coupled with Peptide resin, followed by cleavage, de protection and preparative purification using HPLC resulted in Compound 12. The HPLC purity of Compound 12 was assessed by Method B2.

Mass (LCMS): m/z=1236.56 (MH₄⁴⁺), Calculated Mass=4942.21; HPLC Purity: 97.2% (Method B2), RT=11.703 min.

### Example 18: Synthesis of Compound 13

Compound 13 was prepared by solid phase method as per the analogous process given for Example 12 except here Moiety A-di-tert butyl ester was coupled with Peptide resin, followed by cleavage, de protection and preparative purification using HPLC resulted in Compound 13. The HPLC purity of Compound 13 was assessed by Method B2.

Mass (LCMS): m/z=1579.52 (MH₃³⁻), Calculated Mass=4741.548; HPLC Purity: 96.5% (Method B2), RT=14.76 min.

### Example 19: Synthesis of Compound 14

The parent peptide was synthesized by solid-phase method. The starting resin used for synthesis was Fmoc-Rink amide resin. Selectively de-blocking of Fmoc protected amino group of rink amide resin using piperidine followed by coupling with Fmoc-Lys(IVDde)-OH with the Rink amide resin. The coupling was performed by using DIPC-HOBt to yield Fmoc-Lys(IVDde)-Rink amide Resin, this completes one cycle. Acetic anhydride and diisopropylethyl amine/pyridine was used to terminate/cap the uncoupled amino groups at the end of every amino acid coupling. Selective de-blocking Fmoc of amino group of Fmoc-Lys(IVDde)-Rink amide Resin using piperidine, then coupling with second amino acid i.e., Fmoc-Ser(tBu)—OH using HOBt and DIPC yielded Fmoc-Ser(tBu)-Lys(IVDde)-rink amide Resin. This completes 2nd cycle. As stated earlier Acetic anhydride and diisopropylethyl amine/pyridine was used to terminate the uncoupled amino groups after amino acid coupling.

The above 3 steps, i.e., deblocking of Fmoc-protection of amino acid attached to the resin, coupling of next amino acid residue in sequence with Fmoc-protected amino group and selective Capping, were repeated for remaining 38 amino acid residues. The side chain of the Fmoc-protected amino acids used were protected orthogonally, e.g., hydroxyl group of Serine, Tyrosine or Threonine were protected with tert-butyl(-tBu) group, amino group of Lysine was protected with tert-butyloxycarbonyl (-Boc) and (4,4-dimethyl-2,6-dioxocyclohex-1-ylidene)-3-methylbutyl (IVDde) group respectively and carboxylic acid groups of aspartic acid or glutamic acid were protected with -tBu group, amide group of glutamine and asparagine were protected with trityl (-Trt) group. The above mentioned three steps, i.e., selective capping, deblocking and then coupling with next Fmoc protected amino acid were performed to get Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Tyr(tBu)-Leu-Glu(OtBu)-Lys(Boc)-Ile-Ala-Ala-Gln(Trt)-Glu(OtBu)-Phe-Val-Asn(Trt)-Trp-Leu-Leu-Ala-Gly-Gly- Pro-Ser(tBu)-Ser(tBu)-Gly-Ala-Pro-Pro-Pro-Ser(tBu)-Lys(IVDde)-Rink amide resin.

De-blocking of Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Tyr(tBu)-Leu-Glu(OtBu)-Lys(Boc)-Ile-Ala-Ala-Gln(Trt)-Glu(OtBu)-Phe-Val-Asn(Trt)-Trp-Leu-Leu-Ala-Gly-Gly-Pro- Ser(tBu)-Ser(tBu)-Gly-Ala-Pro-Pro-Pro-Ser(tBu)-Lys(IVDde)-Rink amide resin. using piperidine followed by Boc protection of Peptide resin using Boc anhydride to yield Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Tyr(tBu)-Leu-Glu(OtBu)-Lys(Boc)-Ile-Ala-Ala-Gln(Trt)- Glu(OtBu)-Phe-Val-Asn(Trt)-Trp-Leu-Leu-Ala-Gly-Gly-Pro-Ser(tBu)-Ser(tBu)-Gly-Ala-Pro-Pro-Pro-Ser(tBu)-Lys(IVDde)-Rink amide resin. De-protection of IVDde group of peptide resin using Hydrazine hydrate followed by coupling of moiety B-di-tert butyl ester was performed by using diisopropylcarbodiimide, N-hydroxybenzotriazole (DIPC-HOBt) as coupling reagent in presence of which yielded compound 14 resin.

Boc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Tyr(tBu)-Leu-Glu(OtBu)-Lys(Boc)-Ile-Ala-Ala-Gln(Trt)-Glu(OtBu)-Phe-Val-Asn(Trt)-Trp-Leu-Leu-Ala-Gly-Gly-Pro-Ser(tBu)-Ser(tBu)-Gly- Ala-Pro-Pro-Pro-Ser(tBu)-Lys(NH moiety B-di-tert butyl ester)-Rink amide resin cleavage and de-protection using trifluoroacetic acid with ethane-1,2-dithiol and triisopropylsilane followed by purification through preparative HPLC resulted in Compound 14. The HPLC purity of Compound 14 was assessed by Method B5.

Mass (LCMS): m/z=993.06 (MH₅⁵⁺), Calculated Mass=4960.26; HPLC Purity: 95.8% (Method B5), RT=28.308 min.

### Example 20: Synthesis of Compound 15

Compound 15 was prepared by solid phase method as per the analogous process given for Example 19, wherein for Compound 15 Fmoc-Ser(OMe)—OH was used at position 13 instead of Fmoc-Tyr(tBu) to get Fmoc-Tyr(tBu)-Aib-Glu(OtBu)-Gly-Thr(tBu)-Phe-Thr(tBu)-Ser(tBu)-Asp(OtBu)-Tyr(tBu)-Ser(tBu)-Ile-Ser(OMe)-Leu-Glu(OtBu)-Lys(Boc)-Ile-Ala-Ala-Gln(Trt)-Glu(OtBu)-Phe-Val-Asn(Trt)-Trp-Leu-Leu- Ala-Gly-Gly-Pro-Ser(tBu)-Ser(tBu)-Gly-Ala-Pro-Pro-Pro-Ser(tBu)-Lys(IVDde)-Rink amide resin.

Then coupling with Moiety B-di-tert butyl ester followed by cleavage, de protection and preparative purification using HPLC resulted in Compound 15. The HPLC purity of Compound 15 was assessed by Method B4.

Mass (LCMS): m/z=980.77 (MH₅⁵⁺), Calculated Mass=4898.8; HPLC Purity: 94% (Method B4), RT=41.5 min.

### Example 21: Synthesis of Compound 16

Compound 16 was prepared by solid phase method as per the analogous process given for Example 16 except here Moiety D-di-tert butyl ester was coupled with Peptide resin, followed by cleavage, de protection and preparative purification using HPLC resulted in Compound 16. The HPLC purity of Compound 16 was assessed by Method B2.

Mass (LCMS): m/z=1243.60 (MH₄⁴⁺), Calculated Mass=4970.37; HPLC Purity: 97.5% (Method B2), RT=19.183 min.

### Biological Studies

### Example 22: Reduction of HbA1c in db/db Type 2 Diabetic Mice After Chronic Treatment

The effect of Compound 2 on % HbA1c, Insulin, Triglycerides levels, food consumption and body weight was studied on mice. This study was performed in a type 2 diabetic mouse (db/db) model. The animals were divided into 6 treatment groups (n=8 per group)—a diabetic control group, Compound 2 (4.5 nM/kg, 9 nM/kg and 18 nM/kg), and Tirzepatide (90 nM/kg and 180 nM/kg) treatment group. All the treatments were injected subcutaneously every third day for 10 doses (q3d*10). % HbA1c, Insulin, Triglycerides levels were measured on Day 0, day 14 and day 28. Cumulative food intake from day 0-28 and % Change in body weight compared to day 0 was calculated on day 28. Results are provided in Table 3.

TABLE 3
Effect on HbA1c, Insulin, Triglycerides, food consumption and body wt.

| | | | | | | | Body Weight (%) | Cumulative Food |
|---|---|---|---|---|---|---|---|---|
| | Δ% HbA1c vs. DC | | Triglycerides (mg/dL) | | Insulin (ng/mL) | | Day 28 vs. | Consumption |
| | Day 29 | | Day 28 | | Day 28 | | Baseline | Day 0-28 |
| Treatment Groups | Mean ± SD | | Mean ± SD | | Mean ± SD | | Mean ± SD | Mean ± SD |
| (n = 8) | Day 14 | Day 28 | Day 14 | Day 28 | Day 14 | Day 28 | Day 28 | Day 28 |
| Diabetic | 0 | 0 | 440.4 ± | 450.3 ± | 19.81 ± | 16.74 ± | 2.0 ± | 202.5 ± |
| Control (DC) | | | 67.0 | 32.4 | 12.40 | 10.28 | 1.8 | 14.0 |
| Compound 2, | −2.43*** | −3.64*** | 218.7 ± | 208.9 ± | 34.75 ± | 33.44 ± | −7.0 ± | 112.4 ± |
| 4.5 nM/kg | | | 56.2*** | 62.7*** | 13.16 | 17.67 | 3.2** | 6.3*** |
| Compound 2, | −2.90*** | −4.19*** | 169.8 ± | 147.4 ± | 69.63 ± | 53.87 ± | −8.9 ± | 111.8 ± |
| 9 nM/kg | | | 44.1*** | 16.3*** | 22.42*** | 17.27*** | 4.6***^{##} | 5.1*** |
| Compound 2, | −3.04*** | −4.25*** | 172.4 ± | 144.3 ± | 75.09 ± | 76.93 ± | −9.3 ± | 89.5 ± |
| 18 nM/kg | | | 40.4*** | 20.0*** | 37.48*** | 12.90*** | 6.6***^{##} | 15.5*** |
| Tirzepatide, | −2.63*** | −3.43*** | 168.7 ± | 167.4 ± | 65.23 ± | 52.32 ± | −0.9 ± | 119.1 ± |
| 90 nM/kg | | | 61.7*** | 61.1*** | 16.64*** | 14.90*** | 3.6 | 0.9*** |
| Tirzepatide, | −3.50*** | −3.74*** | 143.4 ± | 141.3 ± | 71.77 ± | 60.19 ± | −6.8 ± | 90.1 ± |
| 180 nM/kg | | | 47.1*** | 32.3*** | 16.62*** | 16.08*** | 3.2*** | 3.4*** |

For % HbA1c, Insulin and Triglyceride Data: Two way ANOVA followed by Bonferroni's post test, where
* = p < 0.05,
**= p < 0.01,
***= p < 0.001 vs Diabetic Control;
# = p < 0.05,
^{##}= p < 0.01,
### = p < 0.001 vs Tirzepatide at 90 nM/kg
For Body weight change and Cumulative food consumption Data: One way ANOVA followed by
Bonferroni's post test, where
* = p < 0.05,
**= p < 0.01,
***= p < 0.001 vs Diabetic Control

As evident from the results, Compound 2 at a dose of 4.5, 9 and 18 nM/kg showed statistically significant change in HbA1c when compared to the diabetic control both on day 14 and on 28^{th} day. The reduction in HbA1c for Compound 2 exceeded the change showed by tirzepatide at 90 nM/kg dose. A similar effect was seen for Compound 2 on insulin levels wherein at a dose of 9 nM/kg it showed a statistically significant increase in insulin levels when compared to the diabetic control group on day 14. The increase in insulin level was maintained even on day 28. In comparison, tirzepatide at 10 times greater dose (90 nM/kg) showed an equivalent effect on insulin levels. Also, the effect on insulin level shown by Compound 2 at a dose of 18 nM/kg was surprising found to be equivalent to the effect shown by tirzepatide at a dose of 180 nM/kg. The insulin level of Compound 2 at 18 nM was maintained with similar level both on days 14 and 28, however, with tirzepatide treatments it was observed that the insulin level on day 28 tended to be slightly lower than on day 14. Compound 2 at a dose of 4.5, 9 and 18 nM/kg showed statistically significant decrease in body weight when compared to the diabetic control group on day 28. Surprisingly, the effect of Compound 2 on body weight reduction was superior to the effect shown by tirzepatide at a dose of 180 nM/kg (20 times greater dose). Compound 2 at the studied dose (4.5, 9 and 18 nm/kg) also showed statistically significant reduction in cumulative food consumption when compared to the diabetic control group during the course of the study. Surprisingly, the effect on food consumption for Compound 2 was equivalent to the effect shown by tirzepatide at a dose which was 10 times the dose of Compound 2. Similarly, Compound 2 at a dose of 4.5, 9 and 18 nM/kg showed statistically significant lowering of triglycerides when compared to the diabetic control group. The effect was maintained with slight improvement on day 28. The efficacy of Compound 2 on lowering of triglycerides level was surprisingly found to be similar to the efficacy shown by tirzepatide at about 20 times the dose of Compound 2. While looking at the effect of Compound 2 on reduction on HbA1c and triglycerides levels it was surprisingly observed that the effect improved on day 28 as compared to day 14. For instance, reduction in HbA1c at 9 nM/kg and 18 nM/kg dose on day 29 was more than 40% than the reduction on day 14. In comparison, tirzepatide at 180 nM/kg dose showed minimal improvement in HbA1c reduction from day 14 to day 28.

### Example 23: cAMP Assay

In-vitro potency determination was performed using cAMP assay. G protein coupled receptor (GPCR) activation following ligand binding initiates a series of second messenger cascades that results in a cellular response. Signaling by the GLP-1R and GIP-R involves activation of adenylate cyclase and cAMP production. Cellular cAMP production was determined using the cAMP Hunter™ eXpress GPCR Assay (Eurofins DiscoveRx).

In cellular cAMP assays, Compound 2 had a half-maximal effective concentration of 4.1 nM on GLP-1R—expressing cells vs about 6.86 nM for Tirzepatide with a Tirzepatide/Compound 2 ratio of 1.68. Also half-maximal effective concentration of Compound 2 was 2.3 nM on GIPR—expressing cells vs 1.89 nM for Tirzepatide with a Tirzepatide/Compound 2 ratio of 0.81.

These results demonstrate that the representative Compound 2 is a potent inhibitor of both GLP-1 and GIP receptor.

### Example 24: Reduction in Blood Glucose and Effect on Body Weight & Food Intake

The effect of Compounds of present invention on blood glucose was studied on mice. This study was performed in a type 2 diabetic mouse (db/db) model. The animals were divided into 8 treatment groups (n=6 per group)—a diabetic control group, Compound 2 to Compound 7 (3 nM/kg) and a Tirzepatide (10 nM/kg) treatment group. Compound 1 (6 nM/kg) and Compound 2 (6 nM/kg) was compared with Tirzepatide (59 nM/kg) in a separate study (treatment n=5). Baseline blood glucose was measured from all the animals. All animals were administered with its respective test item subcutaneously. Blood glucose was measured at 4 hr, 12 hr, 24 hr, 48 hr, 72 hr and 96 hr post treatment. Delta blood glucose (mM) was calculated. Results are provided in Table 4. Similarly body weight changes and cumulative food consumption was measured at 96 hr post treatment. The results are provided in Table 5 below.

TABLE 4
Effect on blood glucose

| | Delta Blood Glucose (mM), Mean (±SD) | | | | | |
|---|---|---|---|---|---|---|
| | 4 hr | 12 hr | 24 hr | 48 hr | 72 hr | 96 hr |
| Treatment Groups | | | | | | |
| (n = 6) | | | | | | |
| Diabetic | 3.4 | 4.6 | 4.8 | 4.6 | 4.9 | 5.1 |
| Control | (±2.6) | (±2.1) | (±2) | (±2.6) | (±2.5) | (±2.5) |
| Compound | −12.6 | −10.6 | −8.3 | −7.3 | −4.7 | −0.3 |
| 2 @ 3 nM | (±2.6)*** | (±4.9)*** | (±1.8)*** | (±2.5)*** | (±4.2)*** | (±3.4)* |
| Compound | −9.2 | −2.9 | 1.6 | 0.7 | 2 | 2.1 |
| 3 @ 3 nM | (±4.8)*** | (±4.6)*** | (±2.3) | (±2.7) | (±3.1) | (±2.9) |
| Compound | −1.1 | 0.3 | 2.3 | 2.3 | −0.1 | 2.4 |
| 4 @ 3 nM | (±2.4)* | (±2.5)* | (±3) | (±2.8) | (±6.1) | (±3.2) |
| Compound | 2.5 | −3.4 | 0.6 | 0.3 | 1.9 | 2.6 |
| 5 @ 3 nM | (±3.6) | (±4 7)*** | (±3.7) | (±5.9) | (±5.9) | (±3.7) |
| Compound | −6.1 | −5.9 | −1.3 | 2.4 | 1.7 | 3.9 |
| 6 @ 3 nM | (±2.8)*** | (±2.9)*** | (±5.3)* | (±3.4) | (±5.6) | (±2.8) |
| Compound | −10.0 | −11.6 | −12.2 | −5.1 | 3 | 3.3 |
| 7@ 3 nM | (±1.7)*** | (±4.1)*** | (±4.8)*** | (±2.8)*** | (±1.7) | (±2.7) |
| Tirzepatide | −8.0 | −5.4 | −10.0 | −2.5 | −1.9 | 1.5 |
| @ 10 nM | (±3.3)*** | (±4.4)*** | (±4.5)*** | (±3.5)*** | (±4.5)** | (±3.1)** |
| Treatment Groups | | | | | | |
| (n = 5) | | | | | | |
| Diabetic | 0.6 | 0.6 | 0.6 | 0.2 | 1.5 | 0.8 |
| Control | (±1.3) | (±1.7) | (±2.2) | (±2.1) | (±1.7) | (±2.6) |
| Compound | −12.7 | −13.3 | −11.1 | −5.3 | 0.1 | 0.1 |
| 1 @ 6 nM | (±2.8)*** | (±5.3)*** | (±4.5)*** | (±2.8) | (±1.0) | (±1.2) |
| Compound | −14.4*** | −18.0*** | −16.9*** | −8.9*** | 0.1 | 2.1 |
| 2 @ 6 nM | (±6.4) | (±2.7) | (±1.7) | (±3.4) | (±3.3) | (±2.1) |
| Tirzepatide | −11.8 | −17.7 | −14.9 | −8.1 | −3.2 | 0.2 |
| @ 59 nM | (±4 .1)*** | (±4.4)*** | (±3.9)*** | (±3.5)** | (±3.8) | (±2.5) |

Two way ANOVA followed by Bonferroni's post test, where
*= p < 0.05,
**= p < 0.01,
***= p < 0.001 vs Diabetic Control

TABLE 5
Effect on body weight and food consumption

| | Body Weight | | Cumulative Food | |
|---|---|---|---|---|
| Treatment | Change (%) 96 hr. | | Intake (g) 0-96 hr | |
| Groups (n = 6) | Mean | SD | Mean | SD |
| Diabetic Control | 4.3 | 1.8 | 22.2 | 3.7 |
| Compound 2 @3 nM/kg | −5*** | 1.7 | 14.6*** | 1.0 |
| Compound 3 @3 nM/kg | −2.5*** | 0.6 | 21.9 | 4.5 |
| Compound 4 @3 nM/kg | −2.3*** | 1.4 | 20.5 | 4.1 |
| Compound 5 @3 nM/kg | −0.5* | 1.2 | 24.7 | 1.2 |
| Compound 6 @3 nM/kg | −3.7*** | 2 | 19.1* | 3.4 |
| Compound 7 @3 nM/kg | −0.1 | 5.4 | 20.9 | 7.3 |
| Tirzepatide @ 10 nM/kg | −3.4*** | 1.8 | 13.3*** | 1.3 |
| Treatment | | | | |
| Groups (n = 5) | | | | |
| Diabetic Control | 0.7 | 0.8 | 21.0 | 4.0 |
| Compound 1@ 6 nM/kg | −0.1* | 1.8 | 19.8* | 3.8 |
| Compound 2@ 6 nM/kg | −0.9 | 0.8 | 18.2 | 1.5 |
| Tirzepatide @ 59 nM/kg | −2.8** | 0.6 | 15.9 | 5.7 |

One way ANOVA followed by Dunnett's posttest, where *= p < 0.05, **= p < 0.01, ***= p < 0.001 vs. Diabetic Control

The effect of Compound 2 and Compound 7 on Blood Glucose was further studied on mice at a dose of 10 nM/kg and 30 nM/kg, respectively, wherein blood glucose was measured at 4 hr, 8 hr, 12 hr, 24 hr, 48 hr and 72 hr post treatment and compared with tirzepatide (90 nM/kg). The results are provided below in Table 6. Similarly body weight changes and cumulative food consumption was measured at 72 hr post treatment. The results are provided in Table 7 below.

TABLE 6
Effect of Compound 2 & 7 on blood glucose

| Treatment Groups | Delta Blood Glucose (mM), Mean(±SD) | | | | | |
|---|---|---|---|---|---|---|
| (n = 8) | 4 hr | 8 hr | 12 hr | 24 hr | 48 hr | 72 hr |
| Diabetic | −1.4 | −0.5 | −0.9 | 0.8 | 2.3 | 1.5 |
| Control | (±4.35) | (±4.73) | (±3.42) | (±2.98) | (±1.31) | (±2.37) |
| Compound 2 | −15.6 | −16.3 | −16.0 | −14.6 | −10.1 | −4.1 |
| @10 nM/kg | (±5.21)*** | (±5.45)*** | (±5.56)*** | (±4.01)*** | (±3.74)*** | (±3.64)* |
| Compound 7 | −11.7 | −12.8 | −14.3 | −14.1 | −10.1 | −0.7 |
| @30 nM/kg | (±5.53)*** | (±4.58)*** | (±4.05)*** | (±6.30)*** | (±5.99)*** | (±0.63) |
| Tirzepatide | −10.3 | −9.4 | −13.6 | −14.9 | −10.1 | −4.0 |
| @90 nM/kg | (±4.38)*** | (±3.86)** | (±3.96)*** | (±2.73)*** | (±4.28)*** | (±4.35)* |

Two way ANOVA followed by Bonferroni's posttest, where
*= p < 0.05,
**= p < 0.01,
***= p < 0.001 vs Diabetic Control

TABLE 7
Effect of Compound 2 & 7 on body weight and food consumption

| | | | Cumulative Food | |
|---|---|---|---|---|
| | % Body wt. change | | Consumption (0-72 hr) | |
| Treatment | 72 hr. vs. 0 hr. | | (g) | |
| Groups (n = 8) | Mean | SD | Mean | SD |
| Diabetic Control | 3.6 | 1.9 | 18.3 | 3.4 |
| Compound 2 @10 nM/kg | −7.2*** | 1.9 | 8.4*** | 2.7 |
| Compound 7 @30 nM/kg | −3.7*** | 2.3 | 8.7*** | 0.4 |
| Tirzepatide @90 nM/kg | −6.7*** | 3.3 | 8.1** | 4.3 |

One way ANOVA followed by Dunnett's posttest, where *= p < 0.05, **= p < 0.01, ***= p < 0.001 vs. Diabetic Control

The results demonstrate that the compounds of present invention can effectively reduce the blood glucose levels in T2D. The results also demonstrate that the compounds of present invention are effective for a long duration. It is surprising to see that the effect of Compound 2 on blood glucose reduction was similar to the effect shown by tirzepatide at a dose of about 9 times higher than the Compound 2 dose. It was also surprising that the efficacy was maintained for 72 hrs. Similarly, the compounds showed a statistically significant reduction in food intake and body weight.

In a separate study, the effects of compound 2, 8, 9 and 10 on blood glucose, food intake and body weight were studied in mice. This study was performed in a type 2 diabetic mouse (db/db) model. The animals were divided into 5 treatment groups (n=6)—a diabetic control group, Compound 2 (10 nM/kg), Compound 8 (10 nM/kg), Compound 9 (10 nM/kg) and Compound 10 (10 nM/kg). Baseline blood glucose was measured from all the animals. All the animals were administered with test items subcutaneously. Blood glucose was measured at 4 hr, 12 hr, 24 hr, 48 hr, 72 hr and 96 hr post treatment. Delta blood glucose (mM) was calculated. The results are shown in Table 8. Body weight changes and cumulative food consumption was measured at 96 hr post treatment. The results are shown in Table 9. Similarly the effect of Compounds 11-15 on blood glucose, food intake and body weight was studied in a separate study except for Compound 13. The results are shown in Table 8 (effect on blood glucose) and Table 9 (effect on body weight and food consumption).

TABLE 8
Effect of Compounds 8, 9, 10, 2, 11, 12, 13, 14, 15 and 16 on blood glucose

| | | Delta Blood Glucose (mM), Mean(±SD) | | | | | |
|---|---|---|---|---|---|---|---|
| | 0 hr | 4 hr | 12 hr | 24 hr | 48 hr | 72 hr | 96 hr |
| Treatment Group | | | | | | | |
| (n = 6) | | | | | | | |
| Diabetic Control | 0.0 | 1.1 | 1.2 | 0.8 | 2.2 | 2.7 | 2.7 |
| | | (±3.0) | (±1.8) | (±2.3) | (±2.0) | (±0.8) | (±2.3) |
| Compound 8, | 0.0 | −7.6*** | −8 3*** | −7.6*** | −3.3** | −0.9 | 1.3 |
| 10 nM/kg/s.c/single | | (±2.9) | (5.5) | (±5.3) | (±3.0) | (±1.0) | (±2.1) |
| dose | | | | | | | |
| Compound 9, | 0.0 | −11.3* | −12.3*** | −3.2 | 0.7 | 0.1 | 1.9 |
| 10 nM/kg/s.c/single | | (±3.2) | (±4.5) | (±3.2) | (±2.7) | (±2.0) | (±2.7) |
| dose | | | | | | | |
| Compound 10, | 0.0 | −8.9*** | −10.8*** | −7.8*** | −0.6 | 0.5 | 1.4 |
| 10 nM/kg/s.c/single | | (±2.5) | (±5.7) | (±6.2) | (±2.1) | (±3.5) | (±3.4) |
| dose | | | | | | | |
| Compound 2, | 0.0 | −13.1*** | −11.7*** | −8.2*** | −6.0** | −2.8 | 0.6 |
| 10 nM/kg/s.c/single | | (±1.7) | (±4.3) | (±2.6) | (±2.7) | (±2.4) | (±0.9) |
| dose | | | | | | | |
| Treatment Group | | | | | | | |
| (n = 6) | | | | | | | |
| Diabetic Control | 0.0 | 1.0 | 4.6 | 2.8 | 5.3 | 4.5 | 5.6 |
| | | (±3.3) | (±3.7) | (±2.0) | (±3.6) | (±5.4) | (±3.5) |
| Compound 11, | 0.0 | −10.5*** | −11.2*** | −14.6*** | −6.4*** | −2.9*** | 1.1 |
| 10 nM/kg/s.c/single | | (±3.6) | (±2.7) | (±4.2) | (±0.9) | (±1.7) | (±2.8) |
| dose | | | | | | | |
| Compound 13 | 0.0 | −7.7*** | −5.6*** | −8.1*** | 3.9 | 4.5 | 7.2 |
| 10 nM/kg/s.c/single | | (±3.4) | (±5.0) | (±4.5) | (±2) | (±1.7) | (±2.9) |
| dose | | | | | | | |
| Treatment Group | | | | | | | |
| (n = 5) | | | | | | | |
| Diabetic Control | 0.0 | 0.2 | 2.2 | 1.7 | 2.5 | 3.6 | 7.0 |
| | | (±2.7) | (±3.3) | (±3.1) | (±2.4) | (±4.0) | (±1.7) |
| Compound 16 | 0.0 | −14.4*** | −12.1*** | −10.8*** | −4.9** | 0.8 | 0.7* |
| 10 nM/kg/s.c/single | | (±2.8) | (±2.9) | (±4.3) | (±2.2) | (±2.6) | (±3.1) |
| dose | | | | | | | |
| Treatment Group | | | | | | | |
| (n = 5) | | | | | | | |
| Diabetic Control | 0.0 | −0.1 | 2.1 | 0.7 | 1.2 | 1.0 | 1.1 |
| | (±0.0) | (±1.2) | (±1.7) | (±4.1) | (±1.8) | (±2.2) | (±2.3) |
| Compound 12, | 0.0 | −14.2*** | −13.0*** | −8.0*** | −5.5* | −2.9 | 1.7 |
| 10 nM/kg/s.c/single | (±0.0) | (±5.2) | (±6.6) | (±4.2) | (±3.3) | (±4.4) | (±3.7) |
| dose | | | | | | | |
| Compound 14, | 0.0 | −16.3*** | −13.4*** | −9.6*** | −9.9*** | −5.9** | −0.6 |
| 10 nM/kg/s.c/single | (±0.0) | (±4.2) | (±3.9) | (±4.6) | (±6.1) | (±3.9) | (±1.0) |
| dose | | | | | | | |
| Compound 15, | 0.0 | −13.6*** | −11.5*** | −5.9* | −2.7 | −1.0 | 1.1 |
| 10 nM/kg/s.c/single | (±0.0) | (±4.4) | (±4.7) | (±3.0) | (±1.8) | (±1.2) | (±3.5) |
| dose | | | | | | | |

*p < 0.05,
**p < 0.01,
***p < 0.001 vs Diabetic Control; Two way ANOVA followed by Bonferroni's post-test.

TABLE 9
Effect of Compounds 8, 9, 10, 2, 11, 12, 14
and 15 on body weight and food consumption

| | | | Cumulative food | |
|---|---|---|---|---|
| | Body Wt. Change (%) | | Intake (g) 96 hr. | |
| Treatment | 96 hr. vs. Baseline | | vs. Baseline | |
| Groups (n = 6) | Mean | SD | Mean | SD |
| Diabetic Control | 4.1 | 3.5 | 25.8 | 4.9 |
| Compound 8, | −2.9*** | 0.6 | 16.6*** | 4.8 |
| 10 nM/kg/s.c/single dose | | | | |
| Compound 9, | 0.1 | 1.5 | 23.0 | 3.0 |
| 10 nM/kg/s.c/single dose | | | | |
| Compound 10, | −4.2*** | 0.9 | 16.2*** | 0.8 |
| 10 nM/kg/s.c/single dose | | | | |
| Compound 2, | −4.2*** | 2.1 | 12.3*** | 2.2 |
| 10 nM/kg/s.c/single dose | | | | |
| Treatment | | | | |
| Groups (n = 6) | | | | |
| Diabetic Control | 1.4 | 0.5 | 24.6 | 4.0 |
| Compound 11, | −4.5*** | 1.6 | 14.1*** | 3.4 |
| 10 nM/kg/s.c/single dose | | | | |
| Treatment | | | | |
| Groups (n = 5) | | | | |
| Diabetic Control | 1.8 | 1.0 | 29.8 | 1.2 |
| Compound 12, | −3.1** | 0.9 | 15.2*** | 0.7 |
| 10 nM/kg/s.c/single dose | | | | |
| Compound 14, | −4.1** | 1.1 | 12.3*** | 1.6 |
| 10 nM/kg/s.c/single dose | | | | |
| Compound 15, | −3.6*** | 0.8 | 14.5*** | 0.2 |
| 10 nM/kg/s.c/single dose | | | | |

*p < 0.05, **p < 0.01, ***p < 0.001 vs. Diabetic Control; One way ANOVA followed by Dunnett's post test.

Compound 2, Compound 8, Compound 9, Compound 10, Compound 11 and Compound 14 showed statistically significant blood glucose reduction post treatment. Also statistically significant reduction in food intake and body weight was observed compared to diabetic control.

The results presented above demonstrate that the compounds of present invention are potent inhibitors of GLP-1 and GIP receptors and can be effective in treatment of type 2 diabetes, diabetes with obesity, obesity and hyperlipidemia.

1. A polypeptide or a pharmaceutically acceptable salt thereof comprising the amino acid sequence:
(Seq. ID 1)
Y-X1-E-G-T-F-T-S-D-Y-S-I-X2-L-Xaa15-K-I-A-Xaa19-
X3-Xaa21-F-V-Xaa24-W-L-X4-A-G-G-P-S-S-G-A-P-P-P-
S-X5-X6-X7-X8-X9-X10-X11
Wherein
X1 is Aib, Ser(OMe) or (D)Ser(OMe);
X2 is Tyr, Ser(OMe), (D)Ser(OMe) or Aib;
X3 is Gln or Lys; wherein, when X3 is Lys, the side chain amino (ε amino) group of Lys is acylated with a moiety:
{—U—W—Y—Z 10
wherein U is —C(O)—CH₂—O—(CH₂)₂—O—(CH₂)₂—NH—] wherein} is the point of attachment with group W;
W is selected from a group consisting of —C(O)—NH—(CH₂)ₚ—NH—], —C(O)—C(CH₃)₂—NH—] and —C(O)—CH₂—O—(CH₂)₂—O—(CH₂)₂—NH—], wherein p is 3 or 4 and wherein] is the point of attachment with group Y;
Y is —C(O)—(CH₂)₂—CH(COOH)NH— and — is the point of attachment with the group Z;
Z is —C(O)—(CH₂)ₙ—COOH or —C(O)—(CH₂)ₙ—CH₃ wherein n is an integer from 14 to 20;
and with a proviso that when X3 is Lys and X2 is Aib, then W is not —C(O)—CH₂—O—(CH₂)₂—O—(CH₂)₂—NH—];
X4 is Leu, Ile or Glu;
X5 is absent, Arg or Lys; wherein, when X5 is Lys, the side chain amino (ε amino) group of Lys is acylated with a moiety:
{—U′—W′—Y′—Z′
wherein U′ is —C(O)—CH₂—O—(CH₂)₂—O—(CH₂)₂—NH—] wherein} is the point of attachment with group W′;
W′ is selected from a group consisting of —C(O)—NH—(CH₂)_{q}—NH—], —C(O)—C(CH₃)₂—NH—] and —C(O)—CH₂—O—(CH₂)₂—O—(CH₂)₂—NH—], wherein q is 3 or 4 and wherein] is the point of attachment with group Y′;
Y′ is —C(O)—(CH₂)₂—CH(COOH)NH— and — is the point of attachment with the group Z′;
Z′ is —C(O)—(CH₂)ₘ—COOH or —C(O)—(CH₂)ₘ—CH₃ wherein m is an integer from 14 to 20;
X6 is absent or Lys;
X7 is absent or Lys;
X8 is absent or Lys;
X9 is absent or Lys;
X10 is absent or Lys;
X11 is absent or Lys;
Xaa15 is Asp or Glu;
Xaa19 is Gln or Ala;
Xaa21 is Ala or Glu;
Xaa24 is Gln or Asn;
wherein the acid group of the C terminal amino acid is a free carboxylic acid group or is amidated as a C-terminal primary amide;
and with a proviso at least one of X3 and X5 is Lys.

2. The polypeptide or pharmaceutically acceptable salt thereof according to claim 1, wherein X1 is Aib.

3. The polypeptide or pharmaceutically acceptable salt thereof according to claim 1, wherein X2 is Aib.

4. The polypeptide or pharmaceutically acceptable salt thereof according to claim 1, wherein X1 and X2 are both Aib.

5. The polypeptide or pharmaceutically acceptable salt thereof according to claim 1, wherein X1 is Aib and X2 is Ser(OMe) or (D)Ser(OMe).

6. The polypeptide or pharmaceutically acceptable salt thereof according to claim 1, wherein X1 is Ser(OMe) or (D)Ser(OMe) and X2 is Aib.

7. The polypeptide or pharmaceutically acceptable salt thereof according to any one of claims 1-6, wherein X4 is Ile.

8. The polypeptide or pharmaceutically acceptable salt thereof according to any one of claims 1-7, wherein X5 is Arg.

9. The polypeptide or pharmaceutically acceptable salt thereof according to claim 1, wherein X1 is Aib and X2 is Tyr.

10. The polypeptide or pharmaceutically acceptable salt thereof according to any one of claims 1, 2 and 5, comprising an amino acid sequence:
(Seq. ID 2)
Y-Aib-E-G-T-F-T-S-D-Y-S-I-Ser(OMe)-L-D-K-I-A-Q-X3-
A-F-V-Q-W-L-X4-A-G-G-P-S-S-G-A-P-P-P-S-X5-X6-X7-
X8-X9-X10-X11.

11. The polypeptide or pharmaceutically acceptable salt thereof according to claim 10, wherein X4 is Ile.

12. The polypeptide or pharmaceutically acceptable salt thereof according to any one of claims 1-2 comprising an amino acid sequence:
(Seq. ID 3)
Y-X1-E-G-T-F-T-S-D-Y-S-I-X2-L-D-K-I-A-Q-X3-A-F-V-
Q-W-L-X4-A-G-G-P-S-S-G-A-P-P-P-S
wherein X1 is Aib; X2 is Ser(OMe) or Aib; X4 is Ile or Glu.

13. The polypeptide or pharmaceutically acceptable salt thereof according to claim 12, wherein X2 is Aib and X4 is Ile.

14. The polypeptide or pharmaceutically acceptable salt thereof according to claim 1, comprising an amino acid sequence:
(Seq. ID 4)
Y-Aib-E-G-T-F-T-S-D-Y-S-I-Aib-L-D-K-I-A-Q-X3-A-F-
V-Q-W-L-Ile-A-G-G-P-S-S-G-A-P-P-P-S;
wherein, X3 is Lys and acetylated with the moiety {—U—W—Y—Z and W is selected from a group consisting of —C(O)—NH—(CH₂)ₚ—NH—] or —C(O)—C(CH₃)₂—NH—] wherein] is the point of attachment with group Y and p is 3 or 4.

15. The polypeptide or pharmaceutically acceptable salt thereof according to claim 1, comprising an amino acid sequence selected from the group consisting of:
i)
(SEQ ID NO 5)
Tyr Aib Glu Gly Thr Phe Thr Ser Asp Tyr Ser Ile
Aib Leu Asp Lys Ile Ala Gln Lys Ala Phe Val Gln
Trp Leu Ile Ala Gly Gly Pro Ser Ser Gly Ala Pro
Pro Pro Ser-NH₂;
ii)
(SEQ ID NO 9)
Tyr Aib Glu Gly Thr Phe Thr Ser Asp Tyr Ser Ile
D-Ser-(OMe) Leu Asp Lys Ile Ala Gln Lys Ala Phe
Val Gln Trp Leu Ile Ala Gly Gly Pro Ser Ser Gly
Ala Pro Pro Pro Ser-NH₂;
iii)
(SEQ ID NO 10)
Tyr Aib Glu Gly Thr Phe Thr Ser Asp Tyr Ser Ile
Ser(OMe) Leu Asp Lys Ile Ala Gln Lys Ala Phe
Val Gln Trp Leu Ile Ala Gly Gly Pro Ser Ser Gly
Ala Pro Pro Pro Ser-NH₂;
iv)
(SEQ ID NO 11)
Tyr Aib Glu Gly Thr Phe Thr Ser Asp Tyr Ser Ile
Aib Leu Asp Lys Ile Ala Gln Lys Ala Phe Val Gln
Trp Leu Ile Ala Gly Gly Pro Ser Ser Gly Ala Pro
Pro Pro Ser Arg;
v)
(SEQ ID NO 12)
Tyr Aib Glu Gly Thr Phe Thr Ser Asp Tyr Ser Ile
Tyr Leu Glu Lys Ile Ala Ala Gln Glu Phe Val Asn
Trp Leu Leu Ala Gly Gly Pro Ser Ser Gly Ala Pro
Pro Pro Ser Lys-NH₂;
vi)
(SEQ ID NO 13)
Tyr Aib Glu Gly Thr Phe Thr Ser Asp Tyr Ser Ile
Ser(OMe) Leu Glu Lys Ile Ala Ala Gln Glu Phe
Val Asn Trp Leu Leu Ala Gly Gly Pro Ser Ser Gly
Ala Pro Pro Pro Ser Lys-NH₂;
vii)
(SEQ ID NO 6)
Tyr D-Ser(OMe) Glu Gly Thr Phe Thr Ser Asp Tyr
Ser Ile Aib Leu Asp Lys Ile Ala Gln Lys Ala Phe
Val Gln Trp Leu Ile Ala Gly Gly Pro Ser Ser Gly
Ala Pro Pro Pro Ser-NH₂;
and
viii)
(SEQ ID NO 7)
Tyr Ser(OMe) Glu Gly Thr Phe Thr Ser Asp Tyr
Ser Ile Aib Leu Asp Lys Ile Ala Gln Lys Ala Phe
Val Gln Trp Leu Ile Ala Gly Gly Pro Ser Ser Gly
Ala Pro Pro Pro Ser-NH₂.

16. The polypeptide of any one of the claims 1-11, wherein X5, X6, X7, X8, X9, X10 and X11 are all absent.

17. The polypeptide or pharmaceutically acceptable salt thereof according to any one of claims 1-16, wherein W is —C(O)—C(CH₃)₂—NH—].

18. The polypeptide or pharmaceutically acceptable salt thereof according to any one of claims 1-16, wherein W is —C(O)—NH—(CH₂)ₚ—NH—] and p is 3 or 4.

19. The polypeptide or pharmaceutically acceptable salt thereof according to any one of claims 1-16, wherein W is —C(O)—NH—(CH₂)₄—NH—].

20. The polypeptide or pharmaceutically acceptable salt thereof according to any one of claims 1-2, 5, 7-11 and 15-16, wherein W is —C(O)—CH₂—O—(CH₂)₂—O—(CH₂)₂—NH—].

21. The polypeptide or pharmaceutically acceptable salt thereof according to any one of claims 1-16, wherein Z is —C(O)—(CH₂)ₙ—COOH and n is 16 or 18.

22. The polypeptide or pharmaceutically acceptable salt thereof according to any one of claims 1-16, wherein W is —C(O)—NH—(CH₂)₄—NH—], Z is —C(O)—(CH₂)ₙ—COOH and n is 18.

23. The polypeptide or pharmaceutically acceptable salt thereof according to any one of claims 1-16, wherein W is —C(O)—C(CH₃)₂—NH—], Z is —C(O)—(CH₂)ₙ—COOH and n is 18.

24. The polypeptide or pharmaceutically acceptable salt thereof according to any one of claims 1-2, 5-12 and 15-16, wherein W is —C(O)—CH₂—O—(CH₂)₂—O—(CH₂)₂—NH—], Z is —C(O)—(CH₂)ₙ—COOH and n is 16.

25. The polypeptide or pharmaceutically acceptable salt thereof according to any one of claims 1-2, 5-12 and 15-16, wherein W is —C(O)—CH₂—O—(CH₂)₂—O—(CH₂)₂—NH—], Z is —C(O)—(CH₂)ₙ—COOH and n is 18.

26. The polypeptide or pharmaceutically acceptable salt thereof according to any one of claims 1-7, 9-11 and 15, wherein W′ is —C(O)—C(CH₃)₂—NH—].

27. The polypeptide or pharmaceutically acceptable salt thereof according to any one of claims 1-7, 9-11 and 15, wherein W′ is —C(O)—NH—(CH₂)_{q}—NH—] and q is 3 or 4.

28. The polypeptide or pharmaceutically acceptable salt thereof according to any one of claims 1-7, 9-11 and 15, wherein W′ is —C(O)—NH—(CH₂)₄—NH—].

29. The polypeptide or pharmaceutically acceptable salt thereof according to any one of claims 1-7, 9-11 and 15, wherein W′ is —C(O)—CH₂—O—(CH₂)₂—O—(CH₂)₂—NH—].

30. The polypeptide or pharmaceutically acceptable salt thereof according to any one of claims 1-7, 9-11 and 15, wherein Z′ is —C(O)—(CH₂)ₘ—COOH and m is 16 or 18.

31. The polypeptide or pharmaceutically acceptable salt thereof according to any one of claims 1-7, 9-11 and 15, wherein W′ is —C(O)—NH—(CH₂)₄—NH—], Z′ is —C(O)—(CH₂)ₘ—COOH and m is 18.

32. The polypeptide or pharmaceutically acceptable salt thereof according to any one of claims 1-7, 9-11 and 15, wherein W′ is —C(O)—C(CH₃)₂—NH—], Z′ is —C(O)—(CH₂)ₘ—COOH and m is 18.

33. The polypeptide or pharmaceutically acceptable salt thereof according to any one of claims 1-7, 9-11 and 15, wherein W′ is —C(O)—CH₂—O—(CH₂)₂—O—(CH₂)₂—NH—], Z′ is —C(O)—(CH₂)ₘ—COOH and m is 16.

34. The polypeptide or pharmaceutically acceptable salt thereof according to any one of claims 1-7, 9-11 and 15, wherein W′ is —C(O)—CH₂—O—(CH₂)₂—O—(CH₂)₂—NH—], Z′ is —C(O)—(CH₂)ₘ—COOH and m is 18.

35. The polypeptide or pharmaceutically acceptable salt thereof according to any one of claims 1-34, wherein —U—W—Y—Z and/or —U′—W′—Y′—Z′ is selected from the group consisting of:

| | |
|---|---|
| 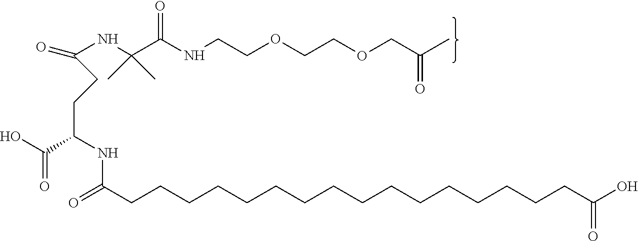 [SMILES: CC(=O)COCCOCCNC(=O)C(C)(C)NC(=O)CC[C@H](NC(=O)CCCCCCCCCCCCCCCCC(=O)O)C(=O)O] | Moiety A; |
| 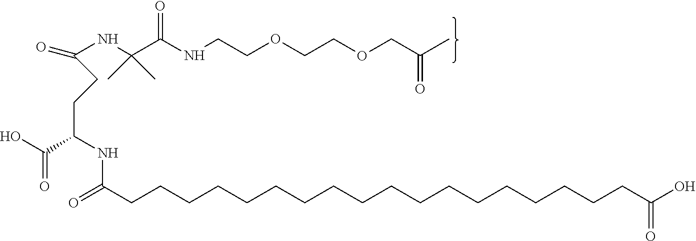 [SMILES: CC(=O)COCCOCCNC(=O)C(C)(C)NC(=O)CC[C@H](NC(=O)CCCCCCCCCCCCCCCCCCC(=O)O)C(=O)O] | Moiety B; |
| 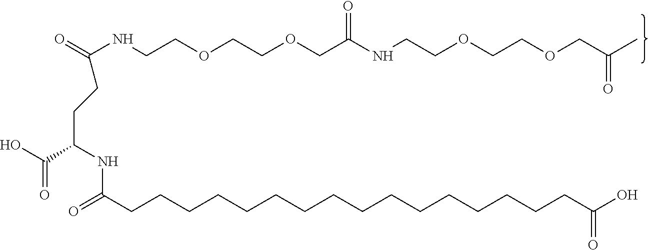 [SMILES: CC(=O)COCCOCCNC(=O)COCCOCCNC(=O)CC[C@H](NC(=O)CCCCCCCCCCCCCCCCC(=O)O)C(=O)O] | Moiety C; |
| 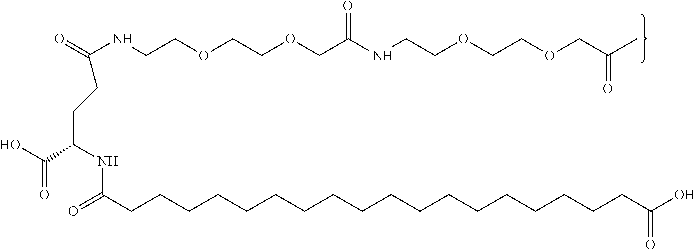 [SMILES: CC(=O)COCCOCCNC(=O)COCCOCCNC(=O)CC[C@H](NC(=O)CCCCCCCCCCCCCCCCCCC(=O)O)C(=O)O] | Moiety D; and |
| 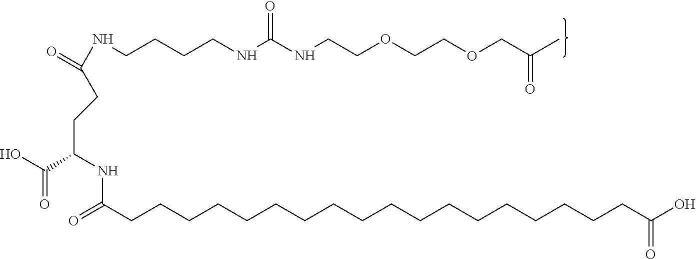 [SMILES: CC(=O)COCCOCCNC(=O)NCCCCNC(=O)CC[C@H](NC(=O)CCCCCCCCCCCCCCCCCCC(=O)O)C(=O)O] | Moiety E |

36. The polypeptide or pharmaceutically acceptable salt thereof according to any one of claims 1-35 wherein the C terminal amino acid is amidated as a C-terminal primary amide.

37. The polypeptide or pharmaceutically acceptable salt thereof according to any one of claims 1-35, wherein the acid group of the C terminal amino acid is a free carboxylic acid.

38. A polypeptide or pharmaceutically acceptable salt thereof, selected from the group consisting of:
Compound 1: 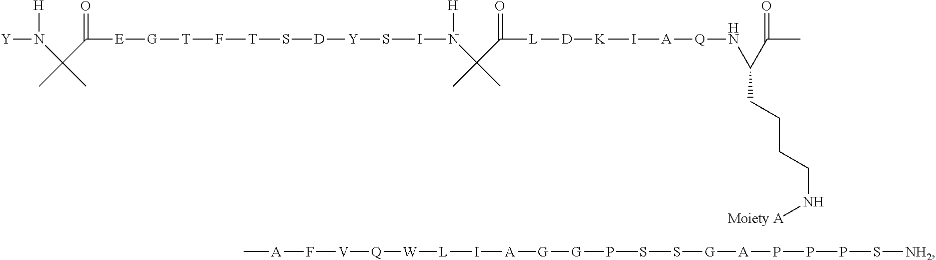
Compound 2 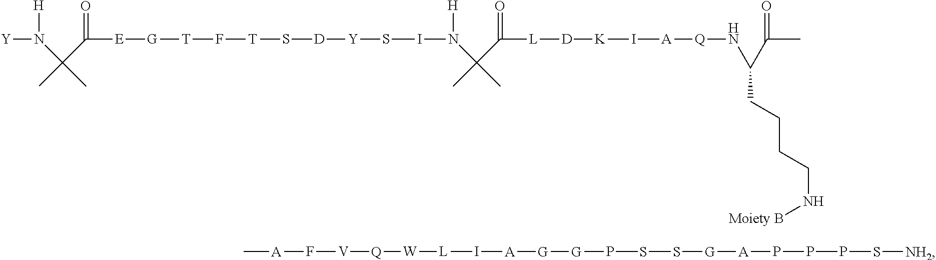
Compound 3 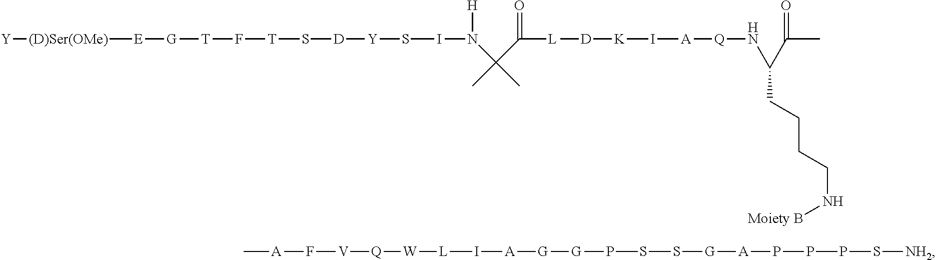
Compound 4 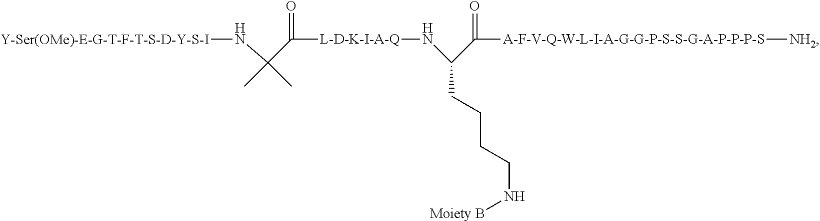
Compound 6 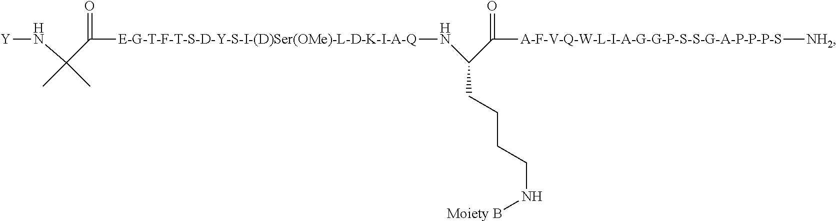
Compound 7 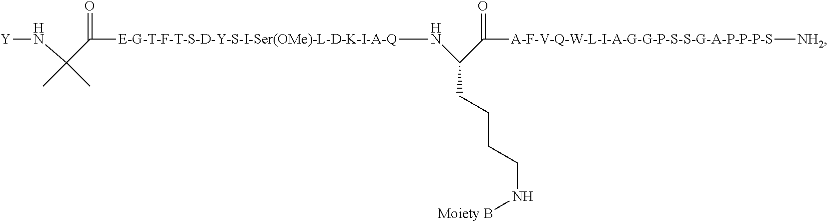
Compound 8 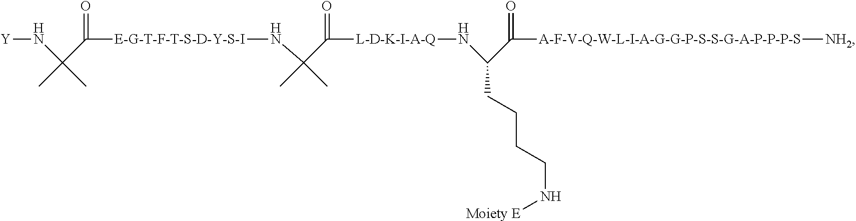
Compound 9 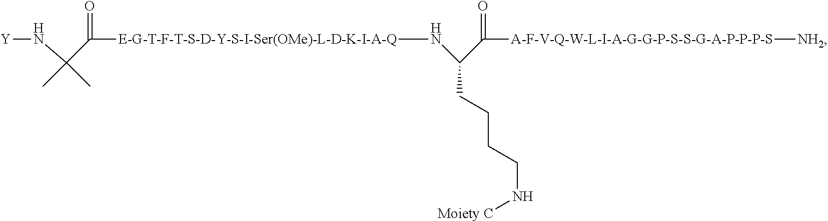
Compound 10 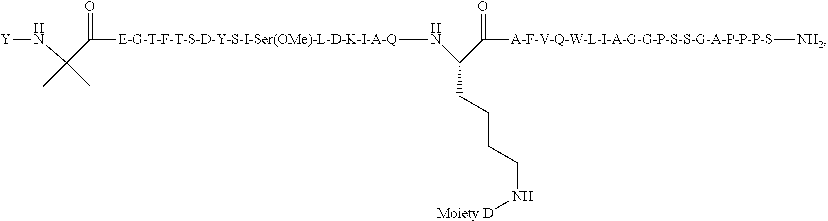
Compound 11 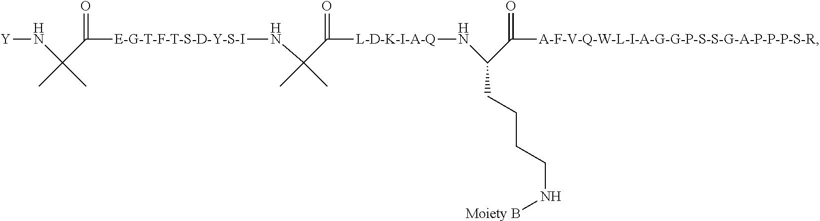
Compound 13 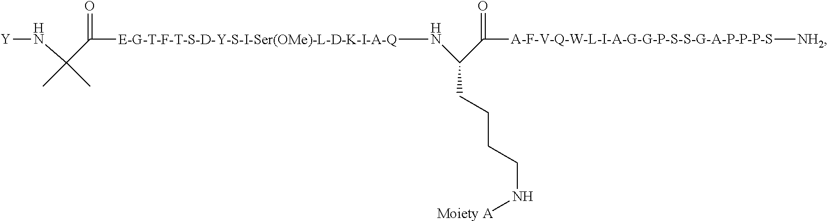
Compound 14 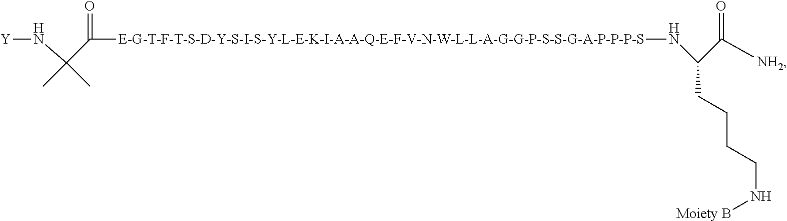
and
Compound 15 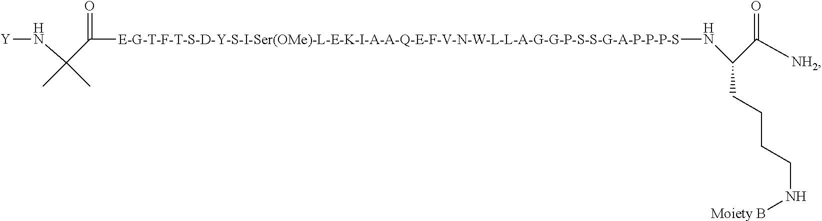
wherein, Moiety A is 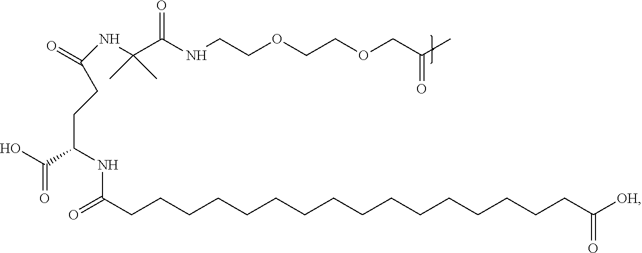 [SMILES: CC(=O)COCCOCCNC(=O)C(C)(C)NC(=O)CC[C@H](NC(=O)CCCCCCCCCCCCCCCCC(=O)O)C(=O)O]
Moiety B is 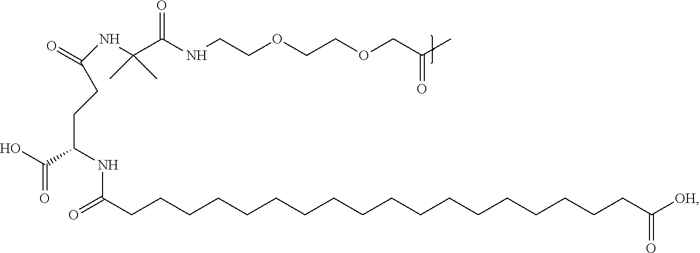 [SMILES: CC(=O)COCCOCCNC(=O)C(C)(C)NC(=O)CC[C@H](NC(=O)CCCCCCCCCCCCCCCCCCC(=O)O)C(=O)O]
Moiety C is 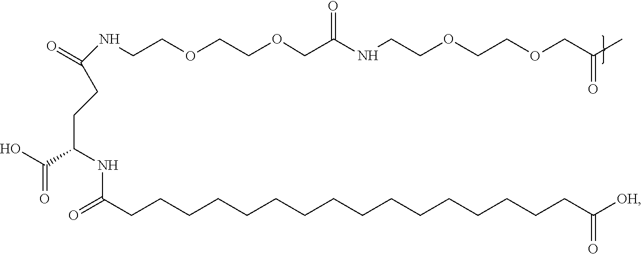 [SMILES: CC(=O)COCCOCCNC(=O)COCCOCCNC(=O)CC[C@H](NC(=O)CCCCCCCCCCCCCCCCC(=O)O)C(=O)O]
Moiety D is 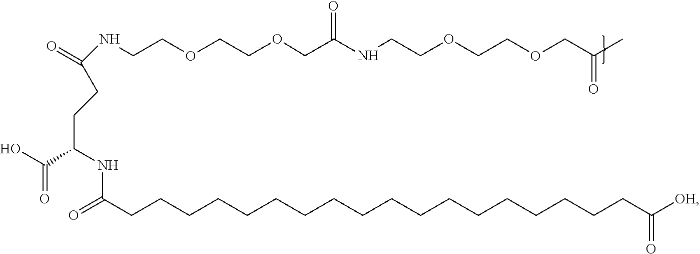 [SMILES: CC(=O)COCCOCCNC(=O)COCCOCCNC(=O)CC[C@H](NC(=O)CCCCCCCCCCCCCCCCCCC(=O)O)C(=O)O]
and
Moiety E is

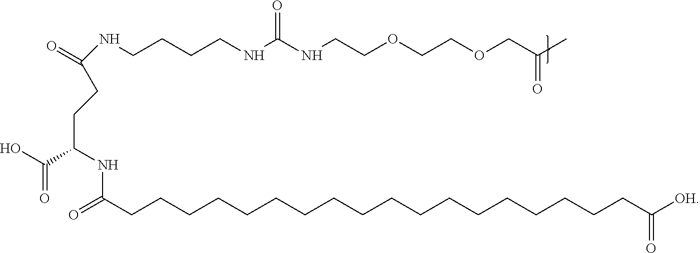 [SMILES: CC(=O)COCCOCCNC(=O)NCCCCNC(=O)CC[C@H](NC(=O)CCCCCCCCCCCCCCCCCCC(=O)O)C(=O)O]

39. A pharmaceutical composition comprising a polypeptide or pharmaceutically acceptable salt thereof according to any one of the claims 1-38, and one or more of a carrier, diluent or pharmaceutically acceptable excipient.

40. A polypeptide or pharmaceutically acceptable salt thereof according to any one of claims 1-38 or a pharmaceutical composition according to claim 39 for use as a medicament.

41. A polypeptide or pharmaceutically acceptable salt thereof according to any one of claims 1-38 or a pharmaceutical composition according to claim 39 for use in the treatment or prevention of a disease in a patient.

42. A polypeptide or pharmaceutically acceptable salt thereof or a pharmaceutical composition for use according to claim 41, wherein said disease is selected from the group consisting of hyperglycemia, type 2 diabetes, impaired glucose tolerance, type 1 diabetes, obesity, hypertension, hyperlipidemia, syndrome X, dyslipidemia, cognitive disorders, atherosclerosis, myocardial infarction, coronary heart disease, stroke, inflammatory bowel syndrome, dyspepsia, alcoholism and gastric ulcers

43. The polypeptide or pharmaceutically acceptable salt thereof or a pharmaceutical composition for use according to claims 40-42 wherein said polypeptide or pharmaceutically acceptable salt thereof or said pharmaceutical composition is provided simultaneously, separately, or sequentially in combination with an effective amount of one or more additional therapeutic agents.

44. A method of treating or preventing hyperglycemia, type 2 diabetes, impaired glucose tolerance, type 1 diabetes, obesity, hypertension, hyperlipidemia, syndrome X, dyslipidemia, cognitive disorders, atherosclerosis, myocardial infarction, coronary heart disease, stroke, inflammatory bowel syndrome, dyspepsia, alcoholism and gastric ulcers in a patient, comprising administering to a patient in need thereof, an effective amount of the polypeptide or pharmaceutically acceptable salt thereof according to any one of claims 1-38.

45. A method of treating or preventing hyperglycemia, type 2 diabetes, impaired glucose tolerance, type 1 diabetes, obesity, hypertension, hyperlipidemia, syndrome X, dyslipidemia, cognitive disorders, atherosclerosis, myocardial infarction, coronary heart disease, stroke, inflammatory bowel syndrome, dyspepsia, alcoholism and gastric ulcers in a patient, wherein said method comprising administering to a patient in need thereof, an effective amount of a pharmaceutical composition according to claim 39.

46. The method according to any one of claims 44-45, further comprising administering simultaneously, separately, or sequentially in combination with an effective amount of one or more therapeutic agents.

47. The polypeptide or pharmaceutically acceptable salt thereof according to any one of claims 1-38, or composition according to claim 39 for use in the preparation of a medicament for the treatment or prevention of hyperglycemia, type 2 diabetes, impaired glucose tolerance, type 1 diabetes, obesity, hypertension, syndrome X, dyslipidemia, cognitive disorders, atherosclerosis, myocardial infarction, coronary heart disease, stroke, inflammatory bowel syndrome, dyspepsia, alcoholism and gastric ulcers.


SEQUENCE LISTING (13 sequences)

SEQ ID NO: 1 - protein, 46 aa, Artificial Sequence
  Synthetic
  MISC_FEATURE: The acid group of the C terminal amino acid is a free carboxylic acid  or is amidated as C-terminal primary amide
  MISC_FEATURE: at least one of X3 and X5 is Lys
  MISC_FEATURE (2)..(2): Xaa = Aib (2-Aminoisobutyric acid), Ser(OMe) or D-Ser(OMe)
  MISC_FEATURE (13)..(13): Xaa = Tyr, Ser(OMe), D-Ser(OMe) or Aib (2-Aminoisobutyric acid)
  MISC_FEATURE (15)..(15): Xaa = Asp or Glu
  MISC_FEATURE (19)..(19): Xaa = Gln or Ala
  MISC_FEATURE (20)..(20): Xaa = Gln or Lys; wherein, when X3 is Lys, the side chain amino(Epsilon amino) group of Lys is substituted
  MISC_FEATURE (21)..(21): Xaa = Ala or Glu
  MISC_FEATURE (24)..(24): Xaa = Gln or Asn
  MISC_FEATURE (27)..(27): Xaa = Leu, Ile or Glu
  MISC_FEATURE (40)..(40): Xaa = absent, Arg or Lys; wherein, when X5 is Lys, the side chain amino (Epsilon amino) group of Lys is substituted
  MISC_FEATURE (41)..(41): Xaa = absent or Lys
  MISC_FEATURE (42)..(42): Xaa = absent or Lys
  MISC_FEATURE (43)..(43): Xaa = absent or Lys
  MISC_FEATURE (44)..(44): Xaa = absent or Lys
  MISC_FEATURE (45)..(45): Xaa = absent or Lys
  MISC_FEATURE (46)..(46): Xaa = absent or Lys
YXEGTFTSDYSIXLXKIAXXXFVXWLXAGGPSSGAPPPSXXXXXXX

SEQ ID NO: 2 - protein, 46 aa, Artificial Sequence
  Synthetic
  MISC_FEATURE: The acid group of the C terminal amino acid is a free carboxylic acid  or is amidated as C-terminal primary amide
  MISC_FEATURE (2)..(2): Xaa = Aib (2-Aminoisobutyric acid)
  MISC_FEATURE (13)..(13): Xaa = Ser(OMe)
  MISC_FEATURE (20)..(20): Xaa = Lys, wherein the side chain amino (Epsilon amino) group of Lys is substituted
  MISC_FEATURE (27)..(27): Xaa = Ile or Glu
  MISC_FEATURE (40)..(40): Xaa = absent or Arg
  MISC_FEATURE (41)..(41): Xaa = absent or Lys
  MISC_FEATURE (42)..(42): Xaa = absent or Lys
  MISC_FEATURE (43)..(43): Xaa = absent or Lys
  MISC_FEATURE (44)..(44): Xaa = absent or Lys
  MISC_FEATURE (45)..(45): Xaa = absent or Lys
  MISC_FEATURE (46)..(46): Xaa = absent or Lys
YXEGTFTSDYSIXLDKIAQXAFVQWLXAGGPSSGAPPPSXXXXXXX

SEQ ID NO: 3 - protein, 39 aa, Artificial Sequence
  Synthetic
  MISC_FEATURE: The acid group of the C terminal amino acid is is a free carboxylic acid  or is amidated as C-terminal primary amide
  MISC_FEATURE (2)..(2): Xaa = Aib (2-Aminoisobutyric acid)
  MISC_FEATURE (13)..(13): Xaa = Ser(OMe) or Aib (2-Aminoisobutyric acid)
  MISC_FEATURE (20)..(20): Xaa = Lys,  wherein the side chain amino (Epsilon amino) group of Lys is substituted
  MISC_FEATURE (27)..(27): Xaa = Ile or Glu
YXEGTFTSDYSIXLDKIAQXAFVQWLXAGGPSSGAPPPS

SEQ ID NO: 4 - protein, 39 aa, Artificial Sequence
  Synthetic
  MISC_FEATURE (2)..(2): Xaa = Aib (2-Aminoisobutyric acid)
  MISC_FEATURE (13)..(13): Xaa = Aib (2-Aminoisobutyric acid)
  MISC_FEATURE (20)..(20): Side chain amino (Epsilon amino) group of Lys is substituted
  MISC_FEATURE (39)..(39): the acid group of the C terminal amino acid is a free carboxylic acid  or is amidated as C-terminal primary amide
YXEGTFTSDYSIXLDKIAQKAFVQWLIAGGPSSGAPPPS

SEQ ID NO: 5 - protein, 39 aa, Artificial Sequence; identical to SEQ ID NO: 4
  Synthetic
  MISC_FEATURE (2)..(2): Xaa = Aib (2-Aminoisobutyric acid)
  MISC_FEATURE (13)..(13): Xaa = Aib (2-Aminoisobutyric acid)
  MISC_FEATURE (20)..(20): Side chain amino (Epsilon amino) group of Lys is substituted
  MISC_FEATURE (39)..(39): Ser at position 39 is amidated as a C-terminal primary amide

SEQ ID NO: 6 - protein, 39 aa, Artificial Sequence; identical to SEQ ID NO: 4
  Synthetic
  MISC_FEATURE (2)..(2): Xaa = D-Ser(OMe)
  MISC_FEATURE (13)..(13): Xaa = Aib (2-Aminoisobutyric acid)
  MISC_FEATURE (20)..(20): Side chain amino (Epsilon amino) group of Lys is substituted
  MISC_FEATURE (39)..(39): Ser at position 39 is amidated as a C-terminal primary amide

SEQ ID NO: 7 - protein, 39 aa, Artificial Sequence; identical to SEQ ID NO: 4
  Synthetic
  MISC_FEATURE (2)..(2): Xaa = Ser (OMe)
  MISC_FEATURE (13)..(13): Xaa = Aib (2-Aminoisobutyric acid)
  MISC_FEATURE (20)..(20): Side chain amino (Epsilon amino) group of Lys is substituted
  MISC_FEATURE (39)..(39): Ser at position 39 is amidated as a C-terminal primary amide

SEQ ID NO: 8 - protein, 39 aa, Artificial Sequence
  Synthetic
  MISC_FEATURE (1)..(1): Xaa = D-Tyr (OEt)
  MISC_FEATURE (2)..(2): Xaa = Aib (2-Aminoisobutyric acid)
  MISC_FEATURE (13)..(13): Xaa = Aib (2-Aminoisobutyric acid)
  MISC_FEATURE (20)..(20): Side chain amino (Epsilon amino) group of Lys is substituted
  MISC_FEATURE (39)..(39): Ser at position 39 is amidated as a C-terminal primary amide
XXEGTFTSDYSIXLDKIAQKAFVQWLIAGGPSSGAPPPS

SEQ ID NO: 9 - protein, 39 aa, Artificial Sequence; identical to SEQ ID NO: 4
  Synthetic
  MISC_FEATURE (2)..(2): Xaa = Aib (2-Aminoisobutyric acid)
  MISC_FEATURE (13)..(13): Xaa = D-Ser (OMe)
  MISC_FEATURE (20)..(20): Side chain amino (Epsilon amino) group of Lys is substituted
  MISC_FEATURE (39)..(39): Ser at position 39 is amidated as a C-terminal primary amide

SEQ ID NO: 10 - protein, 39 aa, Artificial Sequence; identical to SEQ ID NO: 4
  Synthetic
  MISC_FEATURE (2)..(2): Xaa = Aib (2-Aminoisobutyric acid)
  MISC_FEATURE (13)..(13): Xaa = Ser(OMe)
  MISC_FEATURE (20)..(20): Side chain amino (Epsilon amino) group of Lys is substituted
  MISC_FEATURE (39)..(39): Ser at position 39 is amidated as a C-terminal primary amide

SEQ ID NO: 11 - protein, 40 aa, Artificial Sequence
  Synthetic
  MISC_FEATURE (2)..(2): Xaa = Aib (2-Aminoisobutyric acid)
  MISC_FEATURE (13)..(13): Xaa = Aib (2-Aminoisobutyric acid)
  MISC_FEATURE (20)..(20): Side chain amino (Epsilon amino) group of Lys is substituted
YXEGTFTSDYSIXLDKIAQKAFVQWLIAGGPSSGAPPPSR

SEQ ID NO: 12 - protein, 40 aa, Artificial Sequence
  Synthetic
  MISC_FEATURE (2)..(2): Xaa = Aib (2-Aminoisobutyric acid)
  MISC_FEATURE (40)..(40): Side chain amino (Epsilon amino) group of Lys is substituted
  MISC_FEATURE (40)..(40): Lys at position 40 is amidated as a C-terminal primary amide
YXEGTFTSDYSIYLEKIAAQEFVNWLLAGGPSSGAPPPSK

SEQ ID NO: 13 - protein, 40 aa, Artificial Sequence
  Synthetic
  MISC_FEATURE (2)..(2): Xaa = Aib (2-Aminoisobutyric acid)
  misc_feature (13)..(13): Xaa = Ser (OMe)
  MISC_FEATURE (40)..(40): Side chain amino (Epsilon amino) group of Lys is substituted
  MISC_FEATURE (40)..(40): Lys at position 40 is amidated as a C-terminal primary amide
YXEGTFTSDYSIXLEKIAAQEFVNWLLAGGPSSGAPPPSK